(19) United States
(12) Patent Application Publication
(10) Pub. No.: US 2021/0246176 A1
(43) Pub. Date: Aug. 12, 2021

(54) MUCIN-BINDING FUSION PROTEINS

(71) Applicant: Codexis, Inc., Redwood City, CA (US)

(72) Inventors: Marcus Rohovie, Redwood City, CA (US); Nikki Dellas, San Carlos, CA (US); Moulay Hicham Alaoui Ismaili, San Mateo, CA (US)

(21) Appl. No.: 17/155,720

(22) Filed: Jan. 22, 2021

Related U.S. Application Data
(60) Provisional application No. 62/970,851, filed on Feb. 6, 2020.

(51) Int. Cl. C07K 14/335; C07K 14/435; C12N 9/88

(52) U.S. Cl. CPC C07K 14/335; C07K 14/43595; A61K 38/00; C12Y 403/01023; C12N 9/88

(57) ABSTRACT

The present invention provides mucin-binding fusion proteins suitable for extending the residence time of oral therapeutics in the intestinal tract.

The present application claims priority to U.S. Prov. Appln. Ser. No. 62/970,851, filed Feb. 6, 2020, hereby incorporated by reference in its entirety for all purposes.

### FIELD OF THE INVENTION

The present invention provides mucin-binding fusion proteins that extend the residence time of oral therapeutics in the intestinal tract.

### REFERENCE TO SEQUENCE LISTING, TABLE OR COMPUTER PROGRAM

The official copy of the Sequence Listing is submitted concurrently with the specification as an ASCII formatted text file via EFS-Web, with a file name of “CX7-194US1_ST25.txt”, a creation date of Jan. 22, 2021, and a size of 64 kilobytes. The Sequence Listing filed via EFS-Web is part of the specification and is incorporated in its entirety by reference herein.

### BACKGROUND OF THE INVENTION

The gastrointestinal microbial flora is a dense and highly diverse microbial community, that is known to play a significant role in various functions, including metabolism, nutrition, and immune function. In addition, the mucus layer that covers the cells of the gastrointestinal epithelium is important in the maintenance of the homeostasis within the intestinal flora. The mucus layer is biochemically complex, comprised of glycoproteins (e.g., mucins), antimicrobial peptides, immunoglobulins, lipids, electrolytes, and various proteins (See e.g., Etzold et al., Environ. Microbiol., 16:888-903 [2014]; and Johansson et al., Proc. Natl. Acad. Sci., 108 (Suppl. 1): 4659-4665 [2011]). Some bacteria, including members of the Lactobacillus genus, produce mucus-binding proteins that assist these organisms in binding to the intestinal mucus layer and potentiating the retention of the bacteria within the intestinal tract.

### SUMMARY OF THE INVENTION

The present invention provides mucin-binding fusion proteins that extend the residence time of oral therapeutics in the intestinal tract. The present invention provides recombinant fusion proteins comprising a mucin-binding polypeptide and at least one additional polypeptide. In some embodiments of the recombinant fusion proteins, the additional polypeptide comprises an enzyme. In some further embodiments, the enzyme is selected from lyases and decarboxylases. In some additional embodiments, recombinant fusion protein finds use as a therapeutic. In some embodiments, the mucin-binding polypeptide is selected from SEQ ID NO: 2, 4, 6, 8, and 10. In yet some additional embodiments of the recombinant fusion protein, the additional polypeptide comprises a tyrosine ammonia lyase. In some embodiments, the tyrosine ammonia lyase comprises a polypeptide sequence at least 90% identical to SEQ ID NO: 26. In some additional embodiments, the recombinant fusion protein comprises a polypeptide sequence at least 90% identical to a sequence selected from SEQ ID NO: 22 and SEQ ID NO: 24. In some further embodiments, the recombinant fusion protein comprises a polypeptide sequence at least 90% identical to a sequence selected from SEQ ID NO: 12, 14, 16, 18, and 20. The present invention also provides compositions comprising at least one recombinant fusion protein provided herein. The present invention also provides uses for the compositions provided herein.

The present invention also provides recombinant polynucleotide sequences encoding at least one recombinant fusion protein provided herein. In some embodiments, the recombinant polynucleotide sequence is codon-optimized. The present invention also provides expression vectors comprising at least one recombinant polynucleotide sequence provided herein. In some embodiments, the recombinant polynucleotide sequence is operably linked to a control sequence. In some further embodiments, the control sequence is a promoter. In some additional embodiments, the promoter is a heterologous promoter. The present invention also provides host cells comprising at least one expression vector provided herein. In some embodiments, the host cell is eukaryotic or prokaryotic. In some additional embodiments, the host cell is Escherichia coli.

The present invention also provides methods of producing a recombinant mucin-binding fusion protein, comprising culturing at least one host cell provided herein, under conditions that the recombinant mucin-binding fusion protein encoded by the recombinant polynucleotide is produced. In some embodiments, the methods further comprise the step of recovering the recombinant mucin-binding fusion protein. In some additional embodiments, the methods further comprise the step of purifying the recombinant mucin-binding fusion protein.

The present invention also provides pharmaceutical compositions for the treatment of disease, comprising the recombinant mucin-binding fusion protein produced using at least one method provided herein. The present invention further provides pharmaceutical compositions for the treatment of disease, comprising at least one recombinant fusion protein provided herein. In some embodiments, the pharmaceutical composition further comprises a pharmaceutically acceptable carrier and/or excipient.

The present invention also provides methods for treating and/or preventing the symptoms of disease in a subject, comprising providing a subject having disease, and administering at least one pharmaceutical composition provided herein, to the subject. In some embodiments, the pharmaceutical composition is administered orally to the subject. In some additional embodiments of the methods, the subject's symptoms of disease are ameliorated. In some further embodiments of the methods, the subject is able to eat a diet that is less restricted in its specific amino acid content than diets required by subjects exhibiting the symptoms of the disease. In some embodiments, the amino acid is selected from tyrosine, phenylalanine, methionine, and leucine. In some additional embodiments, the subject is an infant or child, while in some alternative embodiments, the subject is an adult or young adult.

### DESCRIPTION OF THE DRAWINGS

FIG. 1 provides a graphic representation of the Lactobacillus reuteri mucin-binding protein (MUB).

FIG. 2 provides a graphic representation of the Lactobacillus acidophilus LBA 1652 mucin-binding protein (MUB).

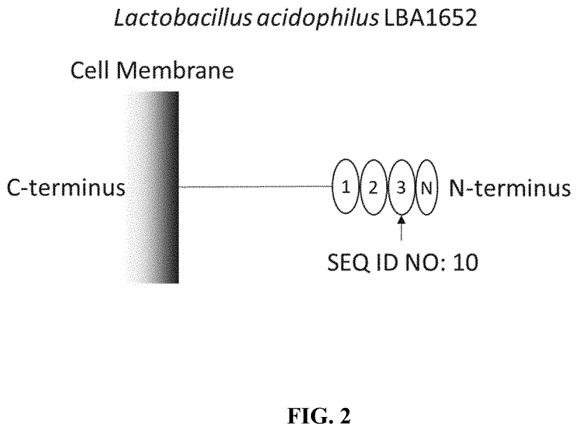

### DESCRIPTION OF THE INVENTION

The present invention provides mucin-binding fusion proteins that extend the residence time of oral therapeutics in the intestinal tract through adhesion to the mucins in the mucus layer. Small intestinal residence times are highly variable and dependent on various factors, but are currently estimated at 4 hr (See e.g., Gao et al., Pharmaceutical Theory and Practice, 455-495 [2017]). The external secreted mucus layer in the small intestines to which mucin-binding fusion proteins adhere, is estimated to renew itself roughly every 4-7 hr, which would give the mucus-binding fusion proteins a potential 7 hr increase in residence time, thereby increasing its efficacy (See e.g., Schneider et al., Sci. Rep., 8: 5760 [2018]).

There are various mucin-binding proteins (MUBs) that have been identified, primarily from members of the Lactobacillus genus but also others such as Lactococcus (See e.g., Boekhorst et al., Microbiol., 152: 273-280 [2006]). The Lactobacillus genus is composed of Gram-positive microaerophilic bacteria that are naturally present as a significant component of the intestinal and colonic flora in humans. They are generally considered to be beneficial for health and are commonly found in probiotics. As probiotics, these organisms may prevent or alleviate infectious diarrhea and promote host resistance to colonization by pathogenic organisms (See e.g., MacKenzie et al., J. Biol. Chem., 284: 32444-32453 [2009]). It is thought that their association with the intestinal mucus through these MUBs increases the retention time of the organisms in the intestinal tract and helps with maintaining normal intestinal function and resistance to pathogens.

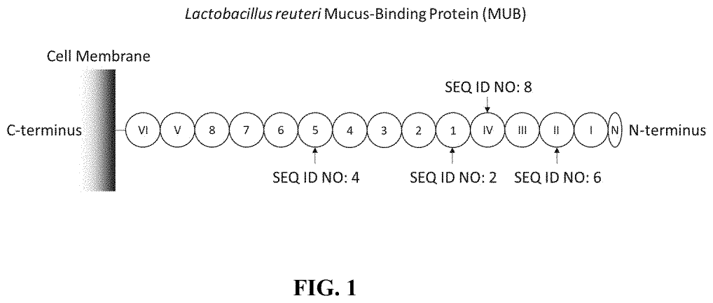

Some MUBs have been characterized and even had fragments crystallized (See e.g., MacKenzie et al., supra, and Etzold et al., Environ. Microbiol., 16(3): 888-903 [2014]). One such MUB from Lactobacillus reuteri is a 174 nm long, 358 kDa fibril-like glycoprotein, containing various domains including two different mucin-binding repeating units present in six and eight copies. These repeats, known as MUB Type 1 and Type 2 repeats respectively, have been shown to be responsible for L. reuteri's adhesion to intestinal mucus. FIG. 1 provides a schematic showing the organization of this MUB, and the domains of interest in the present invention. The two different repeating units are differentiated by Roman numerals (Type 1) and Arabic numerals (Type 2). Type 1 MUB repeats tend to be rather sequence diverse with lengths ranging from 183 to 206 amino acids, while Type 2 MUB repeats tend to have low sequence diversity with a tight range of lengths between 184 and 186 amino acids (See e.g., MacKenzie et al., J. Biol. Chem., 284: 32444-32453 [2009]). This difference in conservation is what defines the types. However, there is very high structural similarity between the two types.

In some embodiments of the present invention, fragments of the L. reuteri MUB or the Lactobacillus acidophilus LBA1652 MUB are fused to other proteins of interest to produce a fusion protein. In some additional embodiments, the proteins fused to the MUB are therapeutic proteins that find use in treating disease. It is contemplated that the MUB portion of the fusion protein increases the retention time of the fusion protein in the intestines. Thus, the therapeutic protein has a longer window of activity in the intestine. In some embodiments, the therapeutic protein is an enzyme. In some embodiments, the fusion protein is administered in a pharmaceutical composition, while in other embodiments, the fusion protein is administered in food, feed, and/or a dietary supplement. It is not intended that the present invention be limited to any particular route or means of administration, as any method that places the fusion protein in the gastrointestinal tract finds use.

### Abbreviations and Definitions

Unless defined otherwise, all technical and scientific terms used herein generally have the same meaning as commonly understood by one of ordinary skill in the art to which this invention pertains. Generally, the nomenclature used herein and the laboratory procedures of cell culture, molecular genetics, microbiology, biochemistry, organic chemistry, analytical chemistry and nucleic acid chemistry described below are those well-known and commonly employed in the art. Such techniques are well-known and described in numerous texts and reference works well known to those of skill in the art. Standard techniques, or modifications thereof, are used for chemical syntheses and chemical analyses. All patents, patent applications, articles and publications mentioned herein, both supra and infra, are hereby expressly incorporated herein by reference.

Although any suitable methods and materials similar or equivalent to those described herein find use in the practice of the present invention, some methods and materials are described herein. It is to be understood that this invention is not limited to the particular methodology, protocols, and reagents described, as these may vary, depending upon the context they are used by those of skill in the art. Accordingly, the terms defined immediately below are more fully described by reference to the application as a whole. All patents, patent applications, articles and publications mentioned herein, both supra and infra, are hereby expressly incorporated herein by reference.

As used herein, the singular “a”, “an,” and “the” include the plural references, unless the context clearly indicates otherwise.

Numeric ranges are inclusive of the numbers defining the range. Thus, every numerical range disclosed herein is intended to encompass every narrower numerical range that falls within such broader numerical range, as if such narrower numerical ranges were all expressly written herein. It is also intended that every maximum (or minimum) numerical limitation disclosed herein includes every lower (or higher) numerical limitation, as if such lower (or higher) numerical limitations were expressly written herein.

The term “about” means an acceptable error for a particular value. In some instances “about” means within 0.05%, 0.5%, 1.0%, or 2.0%, of a given value range. In some instances, “about” means within 1, 2, 3, or 4 standard deviations of a given value.

Furthermore, the headings provided herein are not limitations of the various aspects or embodiments of the invention which can be had by reference to the application as a whole. Accordingly, the terms defined immediately below are more fully defined by reference to the application as a whole. Nonetheless, in order to facilitate understanding of the invention, a number of terms are defined below.

Unless otherwise indicated, nucleic acids are written left to right in 5′ to 3′ orientation; amino acid sequences are written left to right in amino to carboxy orientation, respectively.

As used herein, the term “comprising” and its cognates are used in their inclusive sense (i.e., equivalent to the term “including” and its corresponding cognates).

As used herein, “EC” number refers to the Enzyme Nomenclature of the Nomenclature Committee of the International Union of Biochemistry and Molecular Biology (NC-IUBMB). The IUBMB biochemical classification is a numerical classification system for enzymes based on the chemical reactions they catalyze.

As used herein “ATCC” refers to the American Type Culture Collection whose biorepository collection includes genes and strains.

As used herein “NCBI” refers to National Center for Biological Information and the sequence databases provided therein.

“Protein,” “polypeptide,” and “peptide” are used interchangeably herein to denote a polymer of at least two amino acids covalently linked by an amide bond, regardless of length or post-translational modification (e.g., glycosylation or phosphorylation).

“Amino acids” are referred to herein by either their commonly known three-letter symbols or by the one-letter symbols recommended by IUPAC-IUB Biochemical Nomenclature Commission. Nucleotides, likewise, may be referred to by their commonly accepted single letter codes.

The term “engineered,” “recombinant,” “non-naturally occurring,” and “variant,” when used with reference to a cell, a polynucleotide or a polypeptide refers to a material or a material corresponding to the natural or native form of the material that has been modified in a manner that would not otherwise exist in nature or is identical thereto but produced or derived from synthetic materials and/or by manipulation using recombinant techniques.

Recombinant polypeptides can be produced using any suitable methods known the art. Genes encoding the wild-type polypeptide of interest can be cloned in vectors, such as plasmids, and expressed in desired hosts, such as E. coli, S. cerevisiae, etc. Variants of recombinant polypeptides can be generated by various methods known in the art. Indeed, there is a wide variety of different mutagenesis techniques well known to those skilled in the art. In addition, mutagenesis kits are also available from many commercial molecular biology suppliers. Methods are available to make specific substitutions at defined amino acids (site-directed), specific or random mutations in a localized region of the gene (regio-specific), or random mutagenesis over the entire gene (e.g., saturation mutagenesis). Numerous suitable methods are known to those in the art to generate enzyme variants, including but not limited to site-directed mutagenesis of single-stranded DNA or double-stranded DNA using PCR, cassette mutagenesis, gene synthesis, error-prone PCR, shuffling, and chemical saturation mutagenesis, or any other suitable method known in the art. Non-limiting examples of methods used for DNA and protein engineering are provided in the following patents: U.S. Pat. Nos. 6,117,679; 6,420,175; 6,376,246; 6,586,182; 7,747,391; 7,747,393; 7,783,428; and 8,383,346. After the variants are produced, they can be screened for any desired property (e.g., high or increased activity, or low or reduced activity, increased thermal activity, increased thermal stability, and/or acidic pH stability, etc.). In some embodiments, “recombinant mucin-binding fusion protein polypeptides” (also referred to herein as “engineered mucin-binding fusion protein polypeptides,” “variant mucin-binding fusion protein enzymes,” and “mucin-binding fusion protein variants”) find use.

As used herein, “wild-type” and “naturally-occurring” refer to the form found in nature. For example, a wild-type polypeptide or polynucleotide sequence is a sequence present in an organism that can be isolated from a source in nature and which has not been intentionally modified by human manipulation.

As used herein, “coding sequence” refers to that part of a nucleic acid (e.g., a gene) that encodes an amino acid sequence of a protein.

A “functional fragment” and a “biologically active fragment” are used interchangeably herein refers to a polypeptide that has an amino-terminal and/or carboxy-terminal deletion(s) and/or internal deletions, but where the remaining amino acid sequence is identical to the corresponding positions in the sequence to which it is being compared (e.g., a full-length engineered mucin-binding fusion protein of the present invention) and that retains substantially all of the activity of the full-length polypeptide.

As used herein, “fusion protein” and “chimeric protein” refer to proteins that are generated by joining a least two genes (or portions of genes) that originally coded for separate proteins. In some embodiments, the genes encoding the fusion protein are from the same organism, while in some additional embodiments, the genes encoding the fusion protein are from different organisms. In some embodiments, one portion of the fusion protein is a mucin-binding protein or fragment thereof. In some additional embodiments, one portion of the fusion protein is an enzyme. In some further embodiments, the enzyme comprises a biotherapeutic enzyme. In some additional embodiments, the enzyme finds use in enzyme replacement therapy.

As used herein, “isolated polypeptide” refers to a polypeptide which is substantially separated from other contaminants that naturally accompany it (e.g., protein, lipids, and polynucleotides). The term embraces polypeptides which have been removed or purified from their naturally-occurring environment or expression system (e.g., host cell or in vitro synthesis). The recombinant mucin-binding fusion protein polypeptides may be present within a cell, present in the cellular medium, or prepared in various forms, such as lysates or isolated preparations. As such, in some embodiments, the recombinant mucin-binding fusion protein polypeptides can be an isolated polypeptide.

As used herein, “substantially pure polypeptide” refers to a composition in which the polypeptide species is the predominant species present (i.e., on a molar or weight basis it is more abundant than any other individual macromolecular species in the composition), and is generally a substantially purified composition when the object species comprises at least about 50 percent of the macromolecular species present by mole or % weight. Generally, a substantially pure mucin-binding fusion protein composition comprises about 60% or more, about 70% or more, about 80% or more, about 90% or more, about 95% or more, and about 98% or more of all macromolecular species by mole or % weight present in the composition. In some embodiments, the object species is purified to essential homogeneity (i.e., contaminant species cannot be detected in the composition by conventional detection methods) wherein the composition consists essentially of a single macromolecular species. Solvent species, small molecules (<500 Daltons), and elemental ion species are not considered macromolecular species. In some embodiments, the isolated recombinant mucin-binding fusion protein polypeptides are substantially pure polypeptide compositions.

As used herein, “improved enzyme property” refers to an engineered mucin-binding fusion protein polypeptide that exhibits an improvement in any enzyme property as compared to a reference mucin-binding fusion protein polypeptide and/or as a wild-type mucin-binding fusion protein polypeptide or another engineered mucin-binding fusion protein polypeptide. As used herein, “improved enzyme property” also refers to a mucin-binding fusion protein polypeptide that exhibits an improvement in any enzyme property as compared to the protein to which the MUB was fused when expressed without the MUB. Improved properties include but are not limited to such properties as increased retention time in the intestinal tract, increased expression, increased thermoactivity, increased thermostability, increased pH activity, increased stability, increased enzymatic activity, increased substrate specificity or affinity, increased specific activity, increased resistance to substrate or end-product inhibition, increased chemical stability, improved chemoselectivity, improved solvent stability, increased tolerance to acidic or basic pH, increased tolerance to proteolytic activity (i.e., reduced sensitivity to proteolysis), reduced aggregation, increased solubility, reduced immunogenicity, improved post-translational modification, and altered temperature profile.

As used herein, the terms “protease stable” and “stability to proteolysis” refer to the ability of a protein (e.g., a recombinant mucin-binding fusion protein of the present invention) to function and withstand proteolysis mediated by any proteolytic enzyme or other proteolytic compound or factor. It is not intended that the term be limited to the use of any particular protease to assess the stability of a protein. Indeed, in some embodiments, the engineered mucin-binding fusion proteins of the present invention are stable and retain enzymatic activity in the presence of various proteases. In some additional embodiments, the engineered mucin-binding fusion proteins are stable in the presence of trypsin, chymotrypsin, and/or pepsin. However, it is not intended that the present invention be limited to any specific protease or any particular method of assessing proteolytic stability.

As used herein, the term “pH stability” refers to the ability of a protein (e.g., a recombinant mucin-binding fusion protein of the present invention) to function after incubation at a particular pH. In some embodiments, the present invention provides recombinant mucin-binding fusion proteins that are stable at a range of pHs, including, but not limited to the range of pH 2 to pH 7. In some embodiments, the recombinant mucin-binding fusion proteins are stable at different pH ranges. It is not intended that the present invention be limited to any pH stability level nor pH range.

As used herein, the term “improved tolerance to acidic pH” means that a recombinant mucin-binding fusion protein according to the invention will have increased stability (higher retained activity at about pH 7, 6, 5, 4 3, 2, or even lower, after exposure to acidic pH for a specified period of time [e.g., 1 hour, up to 24 hours, etc.]) as compared to a reference mucin-binding fusion protein or another enzyme. “Physiological pH” as used herein means the pH range generally found in a subject's (e.g., human) blood.

The term “basic pH” (e.g., used with reference to improved stability to basic pH conditions or increased tolerance to basic pH) means a pH range of about 7 to 11.

The term “acidic pH” (e.g., used with reference to improved stability to acidic pH conditions or increased tolerance to acidic pH) means a pH range that encompasses any pH values less than 7. In some embodiments, the acid pH is less than 7, while in some other embodiments, the pH is less than about 6, 5, 4, 3, 2, or lower. In some alternative embodiments, the recombinant mucin-binding fusion proteins of the present invention are stable at pH levels of 2 to 4. However, it is not intended that the present invention be limited to any specific pH value or range of values.

The term “thermal stability” refers to the ability of a protein (e.g., a recombinant mucin-binding fusion protein of the present invention) to function at a particular temperature. Thermal stability can be measured by any method known in the art (e.g., the methods described herein). It is not intended that the present invention be limited to any specific temperature stability level nor temperature range.

The term “chemical stability” refers to the ability of a protein (e.g., a recombinant mucin-binding fusion protein of the present invention) to function in the presence of a chemical that adversely affects the function of another protein. It is not intended that the present invention be limited to any specific chemical stability level nor range of chemical stabilities.

As used herein, a “vector” is a DNA construct for introducing a DNA sequence into a cell. In some embodiments, the vector is an expression vector that is operably linked to a suitable control sequence capable of effecting the expression in a suitable host of the polypeptide encoded in the DNA sequence. In some embodiments, an “expression vector” has a promoter sequence operably linked to the DNA sequence (e.g., transgene) to drive expression in a host cell, and in some embodiments, also comprises a transcription terminator sequence.

As used herein, “codon optimized” refers to changes in the codons of the polynucleotide encoding a protein to those preferentially used in a particular organism such that the encoded protein is more efficiently expressed in the organism of interest. Although the genetic code is degenerate in that most amino acids are represented by several codons, called “synonyms” or “synonymous” codons, it is well known that codon usage by particular organisms is nonrandom and biased towards particular codon triplets. This codon usage bias may be higher in reference to a given gene, genes of common function or ancestral origin, highly expressed proteins versus low copy number proteins, and the aggregate protein coding regions of an organism's genome. In some embodiments, the polynucleotides encoding the mucin-binding fusion protein enzymes may be codon optimized for optimal production from the host organism selected for expression.

As used herein, “control sequence” refers herein to include all components, which are necessary or advantageous for the expression of a polynucleotide and/or polypeptide of the present application. Each control sequence may be native or foreign to the nucleic acid sequence encoding the polypeptide. Such control sequences include, but are not limited to, a leader, polyadenylation sequence, propeptide sequence, promoter sequence, signal peptide sequence, initiation sequence and transcription terminator. At a minimum, the control sequences include a promoter, and transcriptional and translational stop signals. The control sequences may be provided with linkers for the purpose of introducing specific restriction sites facilitating ligation of the control sequences with the coding region of the nucleic acid sequence encoding a polypeptide.

“Operably linked” is defined herein as a configuration in which a control sequence is appropriately placed (i.e., in a functional relationship) at a position relative to a polynucleotide of interest such that the control sequence directs or regulates the expression of the polynucleotide and/or polypeptide of interest.

As used herein, “promoter sequence” refers to a nucleic acid sequence that is recognized by a host cell for expression of a polynucleotide of interest, such as a coding sequence. The promoter sequence contains transcriptional control sequences, which mediate the expression of a polynucleotide of interest. The promoter may be any nucleic acid sequence which shows transcriptional activity in the host cell of choice including mutant, truncated, and hybrid promoters, and may be obtained from genes encoding extracellular or intracellular polypeptides either homologous or heterologous to the host cell.

As used herein, the term “expression” includes any step involved in the production of the polypeptide including, but not limited to, transcription, post-transcriptional modification, translation, and post-translational modification. In some embodiments, the term also encompasses secretion of the polypeptide from a cell.

As used herein, the term “produces” refers to the production of proteins and/or other compounds by cells. It is intended that the term encompass any step involved in the production of polypeptides including, but not limited to, transcription, post-transcriptional modification, translation, and post-translational modification. In some embodiments, the term also encompasses secretion of the polypeptide from a cell.

As used herein, an amino acid or nucleotide sequence (e.g., a promoter sequence, signal peptide, terminator sequence, etc.) is “heterologous” to another sequence with which it is operably linked if the two sequences are not associated in nature.

As used herein, the terms “host cell” and “host strain” refer to suitable hosts for expression vectors comprising DNA provided herein (e.g., the polynucleotides encoding the mucin-binding fusion protein variants). In some embodiments, the host cells are prokaryotic or eukaryotic cells that have been transformed or transfected with vectors constructed using recombinant DNA techniques as known in the art.

The term “analogue” means a polypeptide having more than 70% sequence identity but less than 100% sequence identity (e.g., more than 75%, 78%, 80%, 83%, 85%, 88%, 90%, 91%, 92%, 93%, 94%, 95%, 96%, 97%, 98%, 99% sequence identity) with a reference polypeptide. In some embodiments, analogues mean polypeptides that contain one or more non-naturally occurring amino acid residues including, but not limited, to homoarginine, ornithine and norvaline, as well as naturally occurring amino acids. In some embodiments, analogues also include one or more D-amino acid residues and non-peptide linkages between two or more amino acid residues.

As used herein, the term “culturing” refers to the growing of a population of microbial cells under any suitable conditions (e.g., using a liquid, gel or solid medium).

The term “therapeutic” refers to a compound administered to a subject who shows signs or symptoms of pathology having beneficial or desirable medical effects.

The term “pharmaceutical composition” refers to a composition suitable for pharmaceutical use in a mammalian subject (e.g., human) comprising a pharmaceutically effective amount of an engineered mucin-binding fusion protein polypeptide encompassed by the invention and an acceptable carrier.

The term “effective amount” means an amount sufficient to produce the desired result. One of general skill in the art may determine what the effective amount by using routine experimentation.

The terms “isolated” and “purified” are used to refer to a molecule (e.g., an isolated nucleic acid, polypeptide, etc.) or other component that is removed from at least one other component with which it is naturally associated. The term “purified” does not require absolute purity, rather it is intended as a relative definition.

The term “subject” encompasses mammals such as humans, non-human primates, livestock, companion animals, and laboratory animals (e.g., rodents and lagamorphs). It is intended that the term encompass females as well as males.

As used herein, the term “patient” means any subject that is being assessed for, treated for, or is experiencing disease.

The term “infant” refers to a child in the period of the first month afterbirth to approximately one (1) year of age. As used herein, the term “newborn” refers to child in the period from birth to the 28^{th} day of life. The term “premature infant” refers to an infant born after the twentieth completed week of gestation, yet before full term, generally weighing ˜500 to ˜2499 grams at birth. A “very low birth weight infant” is an infant weighing less than 1500 g at birth.

As used herein, the term “child” refers to a person who has not attained the legal age for consent to treatment or research procedures. In some embodiments, the term refers to a person between the time of birth and adolescence.

As used herein, the term “adult” refers to a person who has attained legal age for the relevant jurisdiction (e.g., 18 years of age in the United States). In some embodiments, the term refers to any fully grown, mature organism. In some embodiments, the term “young adult” refers to a person less than 18 years of age, but who has reached sexual maturity.

As used herein, “composition” and “formulation” encompass products comprising at least one engineered mucin-binding fusion protein of the present invention, intended for any suitable use (e.g., pharmaceutical compositions, dietary/nutritional supplements, feed, etc.).

The terms “administration” and “administering” a composition mean providing a composition of the present invention to a subject (e.g., to a person suffering from the effects of disease).

The term “carrier” when used in reference to a pharmaceutical composition means any of the standard pharmaceutical carrier, buffers, and excipients, such as stabilizers, preservatives, and adjuvants.

The term “pharmaceutically acceptable” means a material that can be administered to a subject without causing any undesirable biological effects or interacting in a deleterious manner with any of the components in which it is contained and that possesses the desired biological activity.

As used herein, the term “excipient” refers to any pharmaceutically acceptable additive, carrier, diluent, adjuvant, or other ingredient, other than the active pharmaceutical ingredient (API; e.g., the engineered mucin-binding fusion protein polypeptides of the present invention). Excipients are typically included for formulation and/or administration purposes.

The term “therapeutically effective amount” when used in reference to symptoms of disease/condition refers to the amount and/or concentration of a compound (e.g., engineered mucin-binding fusion protein polypeptides) that ameliorates, attenuates, or eliminates one or more symptom of a disease/condition or prevents or delays the onset of symptom(s).

The term “therapeutically effective amount” when used in reference to a disease/condition refers to the amount and/or concentration of a composition (e.g., engineered mucin-binding fusion protein polypeptides) that ameliorates, attenuates, or eliminates the disease/condition. In some embodiments, the term is use in reference to the amount of a composition that elicits the biological (e.g., medical) response by a tissue, system, or animal subject that is sought by the researcher, physician, veterinarian, or other clinician.

It is intended that the terms “treating,” “treat” and “treatment” encompass preventative (e.g., prophylactic), as well as palliative treatment.

### Production of Fusion Proteins:

In some embodiments, an isolated polynucleotide encoding any of the engineered mucin-binding fusion protein polypeptides provided herein is manipulated in a variety of ways to provide for expression of the gene and production of the polypeptide. In some embodiments, the polynucleotides encoding the polypeptides are provided as expression vectors, in which one or more control sequences is present to regulate the expression of the polynucleotides and/or polypeptides. Manipulation of the isolated polynucleotide prior to its insertion into a vector may be desirable or necessary depending on the expression vector. The techniques for modifying polynucleotides and nucleic acid sequences utilizing recombinant DNA methods are well known in the art.

In some embodiments, the control sequences include among other sequences, promoters, leader sequences, polyadenylation sequences, propeptide sequences, signal peptide sequences, and transcription terminators. As known in the art, suitable promoters can be selected based on the host cells used. For bacterial host cells, exemplary promoters for directing transcription of the nucleic acid constructs of the present disclosure, include, but are not limited to promoters obtained from the E. coli lac operon, Streptomyces coelicolor agarase gene (dagA), Bacillus subtilis levansucrase gene (sacB), Bacillus licheniformis alpha-amylase gene (amyL), Bacillus stearothermophilus maltogenic amylase gene (amyM), Bacillus amyloliquefaciens alpha-amylase gene (amyQ), Bacillus licheniformis penicillinase gene (penP), Bacillus subtilis xylA and xylB genes, and prokaryotic beta-lactamase gene (See e.g., Villa-Kamaroff et al., Proc. Natl Acad. Sci. USA 75:3727-3731 [1978]), as well as the tac promoter (See e.g., DeBoer et al., Proc. Natl Acad. Sci. USA 80: 21-25 [1983]). Exemplary promoters for filamentous fungal host cells, include promoters obtained from the genes for Aspergillus oryzae TAKA amylase, Rhizomucor miehei aspartic proteinase, Aspergillus niger neutral alpha-amylase, Aspergillus niger acid stable alpha-amylase, Aspergillus niger or Aspergillus awamori glucoamylase, Rhizomucor miehei lipase, Aspergillus oryzae alkaline protease, Aspergillus oryzae triose phosphate isomerase, Aspergillus nidulans acetamidase, and Fusarium oxysporum trypsin-like protease (See e.g., WO 96/00787), as well as the NA2-tpi promoter (a hybrid of the promoters from the genes for Aspergillus niger neutral alpha-amylase and Aspergillus oryzae triose phosphate isomerase), and mutant, truncated, and hybrid promoters thereof. Exemplary yeast cell promoters can be from the genes can be from the genes for Saccharomyces cerevisiae enolase (ENO-1), Saccharomyces cerevisiae galactokinase (GAL1), Saccharomyces cerevisiae alcohol dehydrogenase/glyceraldehyde-3-phosphate dehydrogenase (ADH2/GAP), and Saccharomyces cerevisiae 3-phosphoglycerate kinase. Other useful promoters for yeast host cells are known in the art (See e.g., Romanos et al., Yeast 8:423-488 [1992]). Exemplary promoters for use in mammalian cells include, but are not limited to those from cytomegalovirus (CMV), Simian vacuolating virus 40 (SV40), from Homo sapiens phosphoglycerate kinase, beta actin, elongation factor-Ia or glyceraldehyde-3-phosphate dehydrogenase, or from Gallus gallus' β-actin.

In some embodiments, the control sequence is a suitable transcription terminator sequence, a sequence recognized by a host cell to terminate transcription. The terminator sequence is operably linked to the 3′ terminus of the nucleic acid sequence encoding the polypeptide. Any terminator which is functional in the host cell of choice finds use in the present invention. For example, exemplary transcription terminators for filamentous fungal host cells can be obtained from the genes for Aspergillus oryzae TAKA amylase, Aspergillus niger glucoamylase, Aspergillus nidulans anthranilate synthase, Aspergillus niger alpha-glucosidase, and Fusarium oxysporum trypsin-like protease. Exemplary terminators for yeast host cells can be obtained from the genes for Saccharomyces cerevisiae enolase, Saccharomyces cerevisiae cytochrome C (CYC1), and Saccharomyces cerevisiae glyceraldehyde-3-phosphate dehydrogenase. Other useful terminators for yeast host cells are known in the art (See e.g., Romanos et al., supra). Exemplary terminators for mammalian cells include, but are not limited to those from cytomegalovirus (CMV), Simian vacuolating virus 40 (SV40), or from Homo sapiens growth hormone.

In some embodiments, the control sequence is a suitable leader sequence, a non-translated region of an mRNA that is important for translation by the host cell. The leader sequence is operably linked to the 5′ terminus of the nucleic acid sequence encoding the polypeptide. Any leader sequence that is functional in the host cell of choice may be used. Exemplary leaders for filamentous fungal host cells are obtained from the genes for Aspergillus oryzae TAKA amylase and Aspergillus nidulans triose phosphate isomerase. Suitable leaders for yeast host cells include, but are not limited to those obtained from the genes for Saccharomyces cerevisiae enolase (ENO-1), Saccharomyces cerevisiae 3-phosphoglycerate kinase, Saccharomyces cerevisiae alpha-factor, and Saccharomyces cerevisiae alcohol dehydrogenase/glyceraldehyde-3-phosphate dehydrogenase (ADH2/GAP).

In some embodiments, the control sequence may also be a polyadenylation sequence, a sequence operably linked to the 3′ terminus of the nucleic acid sequence and which, when transcribed, is recognized by the host cell as a signal to add polyadenosine residues to transcribed mRNA. Any polyadenylation sequence which is functional in the host cell of choice may be used in the present invention. Exemplary polyadenylation sequences for filamentous fungal host cells include, but are not limited to those from the genes for Aspergillus oryzae TAKA amylase, Aspergillus niger glucoamylase, Aspergillus nidulans anthranilate synthase, Fusarium oxysporum trypsin-like protease, and Aspergillus niger alpha-glucosidase. Useful polyadenylation sequences for yeast host cells are also known in the art (See e.g., Guo and Sherman, Mol. Cell. Biol., 15:5983-5990 [1995]).

In some embodiments, the control sequence is a signal peptide coding region that codes for an amino acid sequence linked to the amino terminus of a polypeptide and directs the encoded polypeptide into the cell's secretory pathway. The 5′ end of the coding sequence of the nucleic acid sequence may inherently contain a signal peptide coding region naturally linked in translation reading frame with the segment of the coding region that encodes the secreted polypeptide. Alternatively, the 5′ end of the coding sequence may contain a signal peptide coding region that is foreign to the coding sequence.

Any signal peptide coding region that directs the expressed polypeptide into the secretory pathway of a host cell of choice finds use for expression of the engineered mucin-binding fusion protein polypeptides provided herein. Effective signal peptide coding regions for bacterial host cells include the signal peptide coding regions including but not limited to those obtained from the genes for Bacillus NCIB 11837 maltogenic amylase, Bacillus stearothermophilus alpha-amylase, Bacillus licheniformis subtilisin, Bacillus licheniformis beta-lactamase, Bacillus stearothermophilus neutral proteases (nprT, nprS, nprM), and Bacillus subtilis prsA. Further signal peptides are known in the art (See e.g., Simonen and Palva, Microbiol. Rev., 57:109-137 [1993]). In some embodiments, effective signal peptide coding regions for filamentous fungal host cells include, but are not limited to the signal peptide coding regions obtained from the genes for Aspergillus oryzae TAKA amylase, Aspergillus niger neutral amylase, Aspergillus niger glucoamylase, Rhizomucor miehei aspartic proteinase, Humicola insolens cellulase, and Humicola lanuginosa lipase. Useful signal peptides for yeast host cells include, but are not limited to those from the genes for Saccharomyces cerevisiae alpha-factor and Saccharomyces cerevisiae invertase. Useful signal peptides for mammalian host cells include but are not limited to those from the genes for immunoglobulin gamma (IgG).

In some embodiments, the control sequence is a propeptide coding region that codes for an amino acid sequence positioned at the amino terminus of a polypeptide. The resultant polypeptide is referred to as a “proenzyme,” “propolypeptide,” or “zymogen,” in some cases). A propolypeptide can be converted to a mature active polypeptide by catalytic or autocatalytic cleavage of the propeptide from the propolypeptide. The propeptide coding region may be obtained from any suitable source, including, but not limited to the genes for Bacillus subtilis alkaline protease (aprE), Bacillus subtilis neutral protease (nprT), Saccharomyces cerevisiae alpha-factor, Rhizomucor miehei aspartic proteinase, and Myceliophthora thermophila lactase (See e.g., WO 95/33836). Where both signal peptide and propeptide regions are present at the amino terminus of a polypeptide, the propeptide region is positioned next to the amino terminus of a polypeptide and the signal peptide region is positioned next to the amino terminus of the propeptide region.

In some embodiments, regulatory sequences are also utilized. These sequences facilitate the regulation of the expression of the polypeptide relative to the growth of the host cell. Examples of regulatory systems are those that cause the expression of the gene to be turned on or off in response to a chemical or physical stimulus, including the presence of a regulatory compound. In prokaryotic host cells, suitable regulatory sequences include, but are not limited to the lac, tac, and trp operator systems. In yeast host cells, suitable regulatory systems include, but are not limited to the ADH2 system or GAL1 system. In filamentous fungi, suitable regulatory sequences include, but are not limited to the TAKA alpha-amylase promoter, Aspergillus niger glucoamylase promoter, and Aspergillus oryzae glucoamylase promoter.

In another aspect, the present invention also provides a recombinant expression vector comprising a polynucleotide encoding an engineered mucin-binding fusion protein polypeptide, and one or more expression regulating regions such as a promoter and a terminator, a replication origin, etc., depending on the type of hosts into which they are to be introduced. In some embodiments, the various nucleic acid and control sequences described above are joined together to produce a recombinant expression vector which includes one or more convenient restriction sites to allow for insertion or substitution of the nucleic acid sequence encoding the variant mucin-binding fusion protein polypeptide at such sites. Alternatively, the polynucleotide sequence(s) of the present invention are expressed by inserting the polynucleotide sequence or a nucleic acid construct comprising the polynucleotide sequence into an appropriate vector for expression. In creating the expression vector, the coding sequence is located in the vector so that the coding sequence is operably linked with the appropriate control sequences for expression.

The recombinant expression vector may be any vector (e.g., a plasmid or virus), that can be conveniently subjected to recombinant DNA procedures and can result in the expression of the variant mucin-binding fusion protein polynucleotide sequence. The choice of the vector will typically depend on the compatibility of the vector with the host cell into which the vector is to be introduced. The vectors may be linear or closed circular plasmids.

In some embodiments, the expression vector is an autonomously replicating vector (i.e., a vector that exists as an extra-chromosomal entity, the replication of which is independent of chromosomal replication, such as a plasmid, an extra-chromosomal element, a minichromosome, or an artificial chromosome). The vector may contain any means for assuring self-replication. In some alternative embodiments, the vector may be one which, when introduced into the host cell, is integrated into the genome and replicated together with the chromosome(s) into which it has been integrated. Furthermore, a single vector or plasmid or two or more vectors or plasmids which together contain the total DNA to be introduced into the genome of the host cell, or a transposon may be used.

In some embodiments, the expression vector preferably contains one or more selectable markers, which permit easy selection of transformed cells.

A “selectable marker” is a gene the product of which provides for biocide or viral resistance, resistance to heavy metals, prototrophy to auxotrophs, and the like. Examples of bacterial selectable markers include, but are not limited to the dal genes from Bacillus subtilis or Bacillus licheniformis, or markers, which confer antibiotic resistance such as ampicillin, kanamycin, chloramphenicol or tetracycline resistance. Suitable markers for yeast host cells include, but are not limited to ADE2, HIS3, LEU2, LYS2, MET3, TRP1, and URA3. Selectable markers for use in a filamentous fungal host cell include, but are not limited to, amdS (acetamidase), argB (ornithine carbamoyltransferases), bar (phosphinothricin acetyltransferase), hph (hygromycin phosphotransferase), niaD (nitrate reductase), pyrG (orotidine-5′-phosphate decarboxylase), sC (sulfate adenyltransferase), and trpC (anthranilate synthase), as well as equivalents thereof. In another aspect, the present invention provides a host cell comprising a polynucleotide encoding at least one engineered mucin-binding fusion protein polypeptide of the present application, the polynucleotide being operatively linked to one or more control sequences for expression of the engineered mucin-binding fusion protein enzyme(s) in the host cell.

Host cells for use in expressing the polypeptides encoded by the expression vectors of the present invention are well known in the art and include but are not limited to, bacterial cells, such as E. coli, Vibrio fluvialis, Streptomyces and Salmonella typhimurium cells; fungal cells, such as yeast cells (e.g., Saccharomyces cerevisiae and Pichia pastoris [e.g., ATCC Accession No. 201178]); insect cells (e.g., Drosophila S2 and Spodoptera Sf9 cells), plant cells, animal cells (e.g., CHO, COS, and BHK), and human cells (e.g., HEK293T, human fibroblast, THP-1, Jurkat and Bowes melanoma cell lines). Exemplary host cells also include various Escherichia coli strains (e.g., W3110 (ΔfhuA) and BL21).

Accordingly, in another aspect, the present invention provides methods for producing the engineered mucin-binding fusion protein polypeptides, where the methods comprise culturing a host cell capable of expressing a polynucleotide encoding the engineered mucin-binding fusion protein polypeptide under conditions suitable for expression of the polypeptide. In some embodiments, the methods further comprise the steps of isolating and/or purifying the mucin-binding fusion protein polypeptides, as described herein.

Appropriate culture media and growth conditions for the above-described host cells are well known in the art. Polynucleotides for expression of the mucin-binding fusion protein may be introduced into cells by various methods known in the art. Techniques include, among others, electroporation, biolistic particle bombardment, liposome mediated transfection, calcium chloride transfection, calcium chloride transformation, and protoplast fusion.

The engineered mucin-binding fusion protein with the properties disclosed herein can be obtained by subjecting the polynucleotide encoding the naturally occurring or engineered mucin-binding fusion protein polypeptide to mutagenesis and/or directed evolution methods known in the art, and as described herein. An exemplary directed evolution technique is mutagenesis and/or DNA shuffling (See e.g., Stemmer, Proc. Natl. Acad. Sci. USA 91:10747-10751 [1994]; WO 95/22625; WO 97/0078; WO 97/35966; WO 98/27230; WO 00/42651; WO 01/75767 and U.S. Pat. No. 6,537,746). Other directed evolution procedures that can be used include, among others, staggered extension process (StEP), in vitro recombination (See e.g., Zhao et al., Nat. Biotechnol., 16:258-261 [1998]), mutagenic PCR (See e.g., Caldwell et al., PCR Methods Appl., 3:5136-5140 [1994]), and cassette mutagenesis (See e.g., Black et al., Proc. Natl. Acad. Sci. USA 93:3525-3529 [1996]).

Recombinant polypeptides can be produced using any suitable methods known in the art. Genes encoding the wild-type polypeptide of interest can be cloned in vectors, such as plasmids, and expressed in desired hosts, such as E. coli, etc. Variants of recombinant polypeptides can be generated by various methods known in the art. Indeed, there is a wide variety of different mutagenesis techniques well known to those skilled in the art. In addition, mutagenesis kits are also available from many commercial molecular biology suppliers. Methods are available to make specific substitutions at defined amino acids (site-directed), specific or random mutations in a localized region of the gene (regio-specific), or random mutagenesis over the entire gene (e.g., saturation mutagenesis). Numerous suitable methods are known to those in the art to generate enzyme variants, including but not limited to site-directed mutagenesis of single-stranded DNA or double-stranded DNA using PCR, cassette mutagenesis, gene synthesis, error-prone PCR, shuffling, and chemical saturation mutagenesis, or any other suitable method known in the art. Mutagenesis and directed evolution methods can be readily applied to enzyme-encoding polynucleotides to generate variant libraries that can be expressed, screened, and assayed. Any suitable mutagenesis and directed evolution methods find use in the present invention and are well known in the art (See e.g., U.S. Pat. Nos. 5,605,793, 5,811,238, 5,830,721, 5,834,252, 5,837,458, 5,928,905, 6,096,548, 6,117,679, 6,132,970, 6,165,793, 6,180,406, 6,251,674, 6,265,201, 6,277,638, 6,287,861, 6,287,862, 6,291,242, 6,297,053, 6,303,344, 6,309,883, 6,319,713, 6,319,714, 6,323,030, 6,326,204, 6,335,160, 6,335,198, 6,344,356, 6,352,859, 6,355,484, 6,358,740, 6,358,742, 6,365,377, 6,365,408, 6,368,861, 6,372,497, 6,337,186, 6,376,246, 6,379,964, 6,387,702, 6,391,552, 6,391,640, 6,395,547, 6,406,855, 6,406,910, 6,413,745, 6,413,774, 6,420,175, 6,423,542, 6,426,224, 6,436,675, 6,444,468, 6,455,253, 6,479,652, 6,482,647, 6,483,011, 6,484,105, 6,489,146, 6,500,617, 6,500,639, 6,506,602, 6,506,603, 6,518,065, 6,519,065, 6,521,453, 6,528,311, 6,537,746, 6,573,098, 6,576,467, 6,579,678, 6,586,182, 6,602,986, 6,605,430, 6,613,514, 6,653,072, 6,686,515, 6,703,240, 6,716,631, 6,825,001, 6,902,922, 6,917,882, 6,946,296, 6,961,664, 6,995,017, 7,024,312, 7,058,515, 7,105,297, 7,148,054, 7,220,566, 7,288,375, 7,384,387, 7,421,347, 7,430,477, 7,462,469, 7,534,564, 7,620,500, 7,620,502, 7,629,170, 7,702,464, 7,747,391, 7,747,393, 7,751,986, 7,776,598, 7,783,428, 7,795,030, 7,853,410, 7,868,138, 7,783,428, 7,873,477, 7,873,499, 7,904,249, 7,957,912, 7,981,614, 8,014,961, 8,029,988, 8,048,674, 8,058,001, 8,076,138, 8,108,150, 8,170,806, 8,224,580, 8,377,681, 8,383,346, 8,457,903, 8,504,498, 8,589,085, 8,762,066, 8,768,871, 8,849,575, 9,593,326, 9,665,694, 9,684,771, 9,864,833, 9,996,661, and all related US, as well as PCT and non-US counterparts; Ling et al., Anal. Biochem., 254(2):157-78 [1997]; Dale et al., Meth. Mol. Biol., 57:369-74 [1996]; Smith, Ann. Rev. Genet., 19:423-462 [1985]; Botstein et al., Science, 229:1193-1201 [1985]; Carter, Biochem. J., 237:1-7 [1986]; Kramer et al., Cell, 38:879-887 [1984]; Wells et al., Gene, 34:315-323 [1985]; Minshull et al., Curr. Op. Chem. Biol., 3:284-290 [1999]; Christians et al., Nat. Biotechnol., 17:259-264 [1999]; Crameri et al., Nature, 391:288-291 [1998]; Crameri, et al., Nat. Biotechnol., 15:436-438 [1997]; Zhang et al., Proc. Nat. Acad. Sci. U.S.A., 94:4504-4509 [1997]; Crameri et al., Nat. Biotechnol., 14:315-319 [1996]; Stemmer, Nature, 370:389-391 [1994]; Stemmer, Proc. Nat. Acad. Sci. USA, 91:10747-10751 [1994]; WO 95/22625; WO 97/0078; WO 97/35966; WO 98/27230; WO 00/42651; WO 01/75767; and WO 2009/152336, all of which are incorporated herein by reference).

In some embodiments, the present invention provides fusion proteins that are suitable for directed evolution in order to improve their properties. In some embodiments, enzyme variants obtained following mutagenesis treatment are screened by subjecting the enzyme variants to a defined temperature (or other assay conditions) and measuring the amount of enzyme activity remaining after heat treatments or other assay conditions. DNA containing the polynucleotide encoding the mucin-binding fusion protein polypeptide is then isolated from the host cell, sequenced to identify the nucleotide sequence changes (if any), and used to express the enzyme in a different or the same host cell. Measuring enzyme activity from the expression libraries can be performed using any suitable method known in the art (e.g., standard biochemistry techniques, such as HPLC analysis).

For engineered polypeptides of known sequence, the polynucleotides encoding the enzyme can be prepared by standard solid-phase methods, according to known synthetic methods. In some embodiments, fragments of up to about 100 bases can be individually synthesized, then joined (e.g., by enzymatic or chemical litigation methods, or polymerase mediated methods) to form any desired continuous sequence. For example, polynucleotides and oligonucleotides disclosed herein can be prepared by chemical synthesis using the classical phosphoramidite method (See e.g., Beaucage et al., Tetra. Lett., 22:1859-69 [1981]; and Matthes et al., EMBO J., 3:801-05 [1984]), as it is typically practiced in automated synthetic methods. According to the phosphoramidite method, oligonucleotides are synthesized (e.g., in an automatic DNA synthesizer), purified, annealed, ligated and cloned in appropriate vectors.

The produced engineered mucin-binding fusion protein polypeptide can be assessed for any desired improved property (e.g., binding, activity, selectivity, stability, acid tolerance, protease sensitivity, etc.), using any suitable assay known in the art, including but not limited to the assays and conditions described herein.

In some embodiments, any of the engineered mucin-binding fusion protein polypeptides expressed in a host cell are recovered from the cells and/or the culture medium using any one or more of the well-known techniques for protein purification, including, among others, lysozyme treatment, sonication, filtration, salting-out, ultra-centrifugation, and chromatography.

Chromatographic techniques for isolation of the mucin-binding fusion protein polypeptides include, among others, reverse phase chromatography high performance liquid chromatography, ion exchange chromatography, hydrophobic interaction chromatography, gel electrophoresis, and affinity chromatography. Conditions for purifying a particular enzyme depends, in part, on factors such as net charge, hydrophobicity, hydrophilicity, molecular weight, molecular shape, etc., and will be apparent to those having skill in the art. In some embodiments, affinity techniques may be used to isolate the improved variant mucin-binding fusion protein enzymes. In some embodiments utilizing affinity chromatography purification, any antibody which specifically binds the variant mucin-binding fusion protein polypeptide finds use.

In some embodiments utilizing affinity chromatography purification, proteins that bind to mucin-binding fusion protein find use. In still other embodiments utilizing affinity-chromatography purifications, any small molecule that binds to the mucin-binding fusion protein active site finds use. For the production of antibodies, various host animals, including but not limited to rabbits, mice, rats, etc., are immunized by injection with a polypeptide (e.g., a mucin-binding fusion protein variant), or a fragment thereof. In some embodiments, the mucin-binding fusion protein polypeptide or fragment is attached to a suitable carrier, such as BSA, by means of a side chain functional group or linkers attached to a side chain functional group.

In some embodiments, the engineered mucin-binding fusion protein polypeptide is produced in a host cell by a method comprising culturing a host cell (e.g., E. coli, S. cerevisiae, Daucus carota, Nicotiana tabacum, H. sapiens (e.g., HEK293T), or Cricetulus griseus (e.g., CHO)) comprising a polynucleotide sequence encoding an engineered mucin-binding fusion protein polypeptide as described herein under conditions conducive to the production of the engineered mucin-binding fusion protein polypeptide and recovering the engineered mucin-binding fusion protein polypeptide from the cells and/or culture medium.

In some embodiments, once the engineered polypeptides are recovered from the recombinant host cells or cell culture medium, they are further purified by any suitable method(s) known in the art. In some additional embodiments, the purified mucin-binding fusion protein polypeptides are combined with other ingredients and compounds to provide compositions and formulations comprising the engineered mucin-binding fusion protein polypeptide as appropriate for different applications and uses (e.g., pharmaceutical compositions). In some additional embodiments, the purified polypeptides or the formulated mucin-binding fusion protein polypeptides are lyophilized.

### Compositions:

The present invention provides various compositions and formats, including but not limited to those described below. In some embodiments, the present invention provides engineered mucin-binding fusion protein polypeptides suitable for use in pharmaceutical and other compositions, such as dietary/nutritional supplements.

Depending on the mode of administration, these compositions comprising a therapeutically effective amount of an engineered mucin-binding fusion protein according to the invention are in the form of a solid, semi-solid, or liquid. In some embodiments, the compositions include other pharmaceutically acceptable components such as diluents, buffers, excipients, salts, emulsifiers, preservatives, stabilizers, fillers, and other ingredients. Details on techniques for formulation and administration are well known in the art and described in the literature.

In some embodiments, the engineered mucin-binding fusion protein polypeptides are formulated for use in pharmaceutical compositions. Any suitable format for use in delivering the engineered mucin-binding fusion protein polypeptides find use in the present invention, including but not limited to pills, tablets, gel tabs, capsules, lozenges, dragees, powders, soft gels, sol-gels, gels, emulsions, implants, patches, sprays, ointments, liniments, creams, pastes, jellies, paints, aerosols, chewing gums, demulcents, sticks, solutions, suspensions (including but not limited to oil-based suspensions, oil-in water emulsions, etc.), slurries, syrups, controlled release formulations, suppositories, etc. In some embodiments, the engineered mucin-binding fusion protein polypeptides are provided in a format suitable for injection or infusion (i.e., in an injectable formulation). In some embodiments, the engineered mucin-binding fusion protein polypeptides are provided in biocompatible matrices such as sol-gels, including silica-based (e.g., oxysilane) sol-gels. In some embodiments, the engineered mucin-binding fusion protein polypeptides are encapsulated. In some alternative embodiments, the engineered mucin-binding fusion protein polypeptides are encapsulated in nanostructures (e.g., nanotubes, nanotubules, nanocapsules, or microcapsules, microspheres, liposomes, etc.). Indeed, it is not intended that the present invention be limited to any particular delivery formulation and/or means of delivery. It is intended that the engineered mucin-binding fusion protein polypeptides be administered by any suitable means known in the art, including but not limited to oral methods.

In some embodiments, the engineered mucin-binding fusion protein polypeptides are chemically modified by glycosylation, chemical crosslinking reagents, pegylation (i.e., modified with polyethylene glycol [PEG] or activated PEG, etc.) or other compounds (See e.g., Ikeda, Amino Acids 29:283-287 [2005]; U.S. Pat. Nos. 7,531,341, 7,534,595, 7,560,263, and 7,53,653; US Pat. Appln. Publ. Nos. 2013/0039898, 2012/0177722, etc.). Indeed, it is not intended that the present invention be limited to any particular delivery method and/or mechanism.

In some additional embodiments, the engineered mucin-binding fusion protein polypeptides are provided in formulations comprising matrix-stabilized enzyme crystals. In some embodiments, the formulation comprises a cross-linked crystalline engineered mucin-binding fusion protein enzyme and a polymer with a reactive moiety that adheres to the enzyme crystals. The present invention also provides engineered mucin-binding fusion protein polypeptides in polymers.

In some embodiments, compositions comprising the engineered mucin-binding fusion protein polypeptides of the present invention include one or more commonly used carrier compounds, including but not limited to sugars (e.g., lactose, sucrose, mannitol, and/or sorbitol), starches (e.g., corn, wheat, rice, potato, or other plant starch), cellulose (e.g., methyl cellulose, hydroxypropylmethyl cellulose, sodium carboxy-methylcellulose), gums (e.g., arabic, tragacanth, guar, etc.), and/or proteins (e.g., gelatin, collagen, etc.). In some embodiments, the engineered mucin-binding fusion protein polypeptides are administered in combination with other pharmaceutical and/or dietary compositions.

### EXPERIMENTAL

The following Examples, including experiments and results achieved, are provided for illustrative purposes only and are not to be construed as limiting the present invention.

In the experimental disclosure below, the following abbreviations apply: ppm (parts per million); M (molar); mM (millimolar), uM and μM (micromolar); nM (nanomolar); mol (moles); gm and g (gram); mg (milligrams); ug and μg (micrograms); L and l (liter); ml and mL (milliliter); cm (centimeters); mm (millimeters); um and μm (micrometers); sec. (seconds); min(s) (minute(s)); h(s) and hr(s) (hour(s)); U (units); MW (molecular weight); rpm (rotations per minute); ° C. (degrees Centigrade); CDS (coding sequence); DNA (deoxyribonucleic acid); RNA (ribonucleic acid); E. coli W3110 (commonly used laboratory E. coli strain, available from the Coli Genetic Stock Center [CGSC], New Haven, Conn.); MBP and MUB (mucin-binding protein); HPLC (high pressure liquid chromatography); ms (mass spectrometry or mass spectroscopy); SDS-PAGE (sodium dodecyl sulfate polyacrylamide gel electrophoresis); FIOPC (fold improvements over positive control); HTP (high throughput); Sigma-Aldrich (Sigma-Aldrich, St. Louis, Mo.); Abcam (Abcam, Inc., Cambridge, Mass.); Thermo Scientific (part of ThermoFisher Scientific, Waltham, Mass.); Gibco (ThermoFisher Scientific); and ThermoFisher Scientific (Thermo Fisher Scientific, Waltham, Mass.).

### Example 1

### Construction of Expression Constructs for Fusions Containing a Mucin-Binding Polypeptide and Another Protein

Polypeptides that confer mucin-binding attributes were identified in MUB from Lactobacillus reuteri and LBA1652 from Lactobacillus acidophilus (FIGS. 1 & 2). Specifically, the polypeptides identified were “Mub_B2” or “MucBP_2” domains as classified in the Pfam protein family database with the amino acid sequences shown in SEQ ID NO: 2, SEQ ID NO: 4, SEQ ID NO: 6, SEQ ID NO: 8, and SEQ ID NO: 10. Analogs of the nucleic acid sequences for these polypeptides were codon-optimized for expression in E. coli and synthesized based upon the sequence of UniProt Accession No. Q9RGN5 for MUB (GenBank Accession No. AF120104.1) or Q5FIL0 for LBA1652 (GenBank Accession No. CP000033.3). These analogs were fused to the nucleic acid sequence of another protein, and then cloned into an expression vector, using methods known in the art. In some experiments, the vector pCK110900 (See U.S. Pat. No. 7,629,157 and US Pat. Appln. Pubn. 2016/0244787, both of which are hereby incorporated by reference) was used. The mucin-binding polypeptides were expressed under the control of a lac promoter and lacI repressor gene, and found to confer increased residence time in the small intestine for the other protein to which they were fused. The expression vector also contained the P15a origin of replication and the chloramphenicol resistance gene.

### Example 2

### Assaying Mucin-Binding of Fusions Containing Enhanced Green Fluorescent Protein

The mucin-binding polypeptides (SEQ ID NO: 2, SEQ ID NO: 4, SEQ ID NO: 6, SEQ ID NO: 8, and SEQ ID NO: 10) were fused to enhanced green fluorescent protein (EGFP) using methods known in the art, to produce SEQ ID NO: 12, SEQ ID NO: 14, SEQ ID NO: 16, SEQ ID NO: 18, and SEQ ID NO: 20. These fusions were assayed to determine their binding to mucins contained in porcine intestinal mucus, using the assay described by Mariscotti, et al (Mariscotti et al., Intl. J. Med. Microbiol., 304:393-404 [2014]). Briefly, MaxiSorp 96-well plates (NUNC™ [Thermo-Scientific]) were coated with mucin from porcine stomach type II (Sigma-Aldrich) by 18 hr incubation at 4° C., using a 0.5 g/L solution in phosphate-buffered saline (PBS), pH 7.4. After removing the solution, the plates were blocked with 1% (w/v) bovine serum albumin (BSA) in PBS pH 7.4, by incubation at room temperature for 2 hr. After removing the blocking solution, purified fusion proteins comprised of EGFP fused to a mucin-binding polypeptide were added at different concentrations up to 20 uM. After incubating at room temperature for 3 hr, the solutions were removed and the plates washed three times with PBS pH 7.4, containing 0.1% polysorbate-20. Horseradish peroxidase (HRP)-conjugated antibodies (Abcam) that recognize the constructs were added using methods known in the art, and the plates were incubated at 4° C. for 18 hr. After removing the antibody solution, the plates were washed three times with PBS pH 7.4, containing 0.1% polysorbate-20. 3,3′,5,5′-tetramethylbenzidine (TMB), the substrate for HRP, was added. The reaction was stopped with an equal volume of 2M sulfuric acid, and the absorbance at 450 nm (indicated as A₄₅₀ below) was measured to determine the extent of mucin binding. The results are summarized in Table 2.1, below.

TABLE 2.1
Mucoadhesion Properties of Various Sequences

| Sequence | Mucoadhesion¹ at A₄₅₀ |
|---|---|
| SEQ ID NO: 12 | − |
| SEQ ID NO: 14 | ++ |
| SEQ ID NO: 16 | + |
| SEQ ID NO: 18 | − |
| SEQ ID NO: 20 | ++ |

¹In this Table, “−” indicates A₄₅₀ values less than 1; “+” indicates values between 1 and 2; and “++” indicates values over 2.

### Example 3

Assaying Mucin-Binding of Fusions Containing an Evolved Tyrosine Ammonia Lyase

The mucin-binding polypeptides of SEQ ID NO: 6 and SEQ ID NO: 10 were fused to an evolved tyrosine ammonia lyase from Stanieria cyanosphaera (SEQ ID NO: 26), using methods known in the art to produce SEQ ID NO: 22 and SEQ ID NO: 24. These fusions were assayed to determine their binding to mucins contained in porcine intestinal mucus using the assay described in Example 2. The results are summarized in Table 3.1, below.

TABLE 3.1
Mucoadhesion Properties of SEQ ID NOS: 22 and 24

| Sequence | Mucoadhesion¹ at A₄₅₀ |
|---|---|
| SEQ ID NO: 22 | + |
| SEQ ID NO: 24 | + |

¹In this Table, “−” indicates A₄₅₀ values less than 1; “+” indicates values between 1 and 2; and “++” indicates values over 2.

All publications, patents, patent applications and other documents cited in this application are hereby incorporated by reference in their entireties for all purposes to the same extent as if each individual publication, patent, patent application or other document were individually indicated to be incorporated by reference for all purposes.

While various specific embodiments have been illustrated and described, it will be appreciated that various changes can be made without departing from the spirit and scope of the invention.

What is claimed is:

1. A recombinant fusion protein comprising a mucin-binding polypeptide and at least one additional polypeptide.

2. The recombinant fusion protein of claim 1, wherein said additional polypeptide comprises an enzyme.

3. The recombinant fusion protein of claim 2, wherein said enzyme is selected from lyases and decarboxylases.

4. The recombinant fusion protein of claim 1, wherein said recombinant fusion protein finds use as a therapeutic.

5. The recombinant fusion protein of claim 1, wherein said mucin-binding polypeptide is selected from SEQ ID NO: 2, 4, 6, 8, and 10.

6. The recombinant fusion protein of claim 1, wherein said additional polypeptide comprises a tyrosine ammonia lyase.

7. The recombinant fusion protein of claim 6, wherein said tyrosine ammonia lyase comprises a polypeptide sequence at least 90% identical to SEQ ID NO: 26.

8. The recombinant fusion protein of claim 7, wherein said recombinant fusion protein comprises a polypeptide sequence at least 90% identical to a sequence selected from SEQ ID NO: 22 and SEQ ID NO: 24.

9. The recombinant fusion protein of claim 5, wherein said recombinant fusion protein comprises a polypeptide sequence at least 90% identical to a sequence selected from SEQ ID NO: 12, 14, 16, 18, and 20.

10. A composition comprising at least one recombinant fusion protein of claim 1.

11. A recombinant polynucleotide sequence encoding at least one recombinant fusion protein set forth in claim 1.

12. The recombinant polynucleotide sequence of claim 11, wherein said polynucleotide sequence is codon-optimized.

13. An expression vector comprising at least one recombinant polynucleotide sequence of claim 11.

14. The expression vector of claim 13, wherein said recombinant polynucleotide sequence is operably linked to a control sequence.

15. The expression vector of claim 13, wherein said control sequence is a promoter.

16. The expression vector of claim 15, wherein said promoter is a heterologous promoter.

17. A host cell comprising at least one expression vector set forth in claim 13.

18. The host cell of claim 17, wherein said host cell is eukaryotic or prokaryotic.

19. The host cell of claim 18, wherein said host cell is Escherichia coli.

20. A method of producing a recombinant mucin-binding fusion protein, comprising culturing said host cell of claim 18, under conditions that said recombinant mucin-binding fusion protein encoded by said recombinant polynucleotide is produced.

21. The method of claim 20, further comprising the step of recovering said recombinant mucin-binding fusion protein.

22. The method of claim 21, further comprising the step of purifying said recombinant mucin-binding fusion protein.

23. A pharmaceutical composition for the treatment of disease, comprising at least one recombinant mucin-binding fusion protein produced using the method of claim 20.

24. A pharmaceutical composition for the treatment of disease, comprising the composition of claim 10.

25. The pharmaceutical composition of claim 24, further comprising a pharmaceutically acceptable carrier and/or excipient.

26. A method for treating and/or preventing the symptoms of disease in a subject, comprising providing a subject having disease, and administering the pharmaceutical composition of any claim 24, to said subject.

27. The method of claim 26, wherein said pharmaceutical composition is administered orally to said subject.

28. The method of claim 26, wherein said symptoms of disease are ameliorated.

29. The method of claim 26, wherein said subject is able to eat a diet that is less restricted in its specific amino acid content than diets required by subjects exhibiting the symptoms of said disease.

30. The method of claim 29, wherein said amino acid is selected from tyrosine, phenylalanine, methionine, and leucine.

31. The method of claim 26, wherein said subject is an infant or child.

32. The method of claim 26, wherein said subject is an adult or young adult.


SEQUENCE LISTING (26 sequences)

SEQ ID NO: 1 - DNA, 597 bp, Artificial Sequence
  fragment of mucin-binding protein from Lactobacillus reuteri
atgcatcaccatcaccatcacggtggcagcggtatggatgttcagcatgcggtgattaactatatcgacggcgaaagcgatgagattctgcacaccgacaaagtcaatggtcattccgatgaaaagatcaactactctacggccgatatgatcaaacaactcgaagcaaaagggtatgagctgtttaaagacaatttcccggctggcgaaaagtttgataacgacgatactaatgatcagttctacaccgtgatttttaaacaccaccgcgaaaacgtagacccaaatcatagttcggcggatggtacgaaaggcaccaagaccttgacagaaacggtccactataaatacgccaacggaactaaagcagcagaggatcagacagcccaagttaccttcacccgtaacggtgtgctggacgacgttacgggcattgtagcttgggggaaatggaatgaagcctcacagagctataaagctttaacgagtccgaccatcgcaggttatgcccctagcgaagctgtggtgaaacgctcgtccaactctgatgcagaacagggtccgaccctgaccgtcatttacactgccgactaatag

SEQ ID NO: 2 - protein, 197 aa, Artificial Sequence
  fragment of mucin-binding protein from Lactobacillus reuteri
MHHHHHHGGSGMDVQHAVINYIDGESDEILHTDKVNGHSDEKINYSTADMIKQLEAKGYELFKDNFPAGEKFDNDDTNDQFYTVIFKHHRENVDPNHSSADGTKGTKTLTETVHYKYANGTKAAEDQTAQVTFTRNGVLDDVTGIVAWGKWNEASQSYKALTSPTIAGYAPSEAVVKRSSNSDAEQGPTLTVIYTAD

SEQ ID NO: 3 - DNA, 591 bp, Artificial Sequence
  fragment of mucin-binding protein from Lactobacillus reuteri
atgcatcaccatcaccatcacggtggcagcggtatgcagaaagttcatgtgcaatatattgatggcgaaaccgaccagatgctgcgccaggatgatcttgatggttacacggacgagacaatcccgtatagcactgcggaagggattaagaagtttgaaggcgatggttacgagctgttcaaagacaactttcctgccggcgagaaattcgacaatgatgataccaacgaccagttctacaccgtcatctttaaacaccaccgtgaaaatgtagatccgaaccatagtagtgcagacggtacgaaaggaaccaagacgttgactgaaaccgtgcactataaatatgctaatggcacaaaagcggcggaagatcaaaccgcccaggttacgtttacacgcaacggcgtgctggatgatgtcaccggtattgtagcatgggggaaatggaatgaagcctcccagtcgtacaaagcgttaacttcaccaaccatcgctggttatgcaccgtctgaagcggtggtgaaacgtagcagcaactctgacgccgaacaaggcccaacgctgaccgtcatttatacggctgactaatag

SEQ ID NO: 4 - protein, 195 aa, Artificial Sequence
  fragment of mucin-binding protein from Lactobacillus reuteri
MHHHHHHGGSGMQKVHVQYIDGETDQMLRQDDLDGYTDETIPYSTAEGIKKFEGDGYELFKDNFPAGEKFDNDDTNDQFYTVIFKHHRENVDPNHSSADGTKGTKTLTETVHYKYANGTKAAEDQTAQVTFTRNGVLDDVTGIVAWGKWNEASQSYKALTSPTIAGYAPSEAVVKRSSNSDAEQGPTLTVIYTAD

SEQ ID NO: 5 - DNA, 630 bp, Artificial Sequence
  fragment of mucin-binding protein from Lactobacillus reuteri
atgcatcaccatcaccatcacggtggcagcggtatgcaagaagcggcgattaacttctatgatgaaaccggccataaactgctggacaatcagacgatccacttgaccggtaagactggcgagaaagtggatcgtacacaggcagatcagacgcttgccgacctggttaaacaaggttacgtcctcgacaaagaaaacaccgctaaagcatttccggccgatgcggtgtatgacaataatgatcagactccacaggagtttaccatttacctgaagcatgggacgacgcacacagatgctaccagcagcaaagccgatcagaagacggtatcggaaaccatccattacgtctataaagacggagttaacgcgaacaaaccggtggcagatgatgcgaataccaccgtgactttcaaacgcggctatacaaccgacaaagtaaccggcaagattgtctcctacgatccttggactgtggacggtaaacaagcggacagtaagacctttgatgccgtgaaatctccggttatcgctgggtatacggcagatcaagcggaagttgcagcccagacggtaactccggattcacagaacattaacaagaccgtctattatacagccgactaatag

SEQ ID NO: 6 - protein, 208 aa, Artificial Sequence
  fragment of mucin-binding protein from Lactobacillus reuteri
MHHHHHHGGSGMQEAAINFYDETGHKLLDNQTIHLTGKTGEKVDRTQADQTLADLVKQGYVLDKENTAKAFPADAVYDNNDQTPQEFTIYLKHGTTHTDATSSKADQKTVSETIHYVYKDGVNANKPVADDANTTVTFKRGYTTDKVTGKIVSYDPWTVDGKQADSKTFDAVKSPVIAGYTADQAEVAAQTVTPDSQNINKTVYYTAD

SEQ ID NO: 7 - DNA, 609 bp, Artificial Sequence
  fragment of mucin-binding protein from Lactobacillus reuteri
atgcatcaccatcaccatcacggtggcagcggtatgcaggaagcacaagcgatcttctatgatgaaaccaccggcaaagagattagcggtacgcgcgaaattgccacagggaagactgacgagaccatctcgtttacgaaagatccgaacgaagtggtgaaagaactggagaaacagggctacgtctttgataaagacaatgcgaagaacaacgtatttgttgctggtaccgcctatgataagaatagtgaagtccatcagtacttcaaatattatttgaaacacggtcatgcgactgttaccccagaccaagacccgcagaaaggccagaagacggtgacacaaaccattaaatacgaatatgcagatggaactgccaccggtctggcggataatgttcagacgcttacgttcaaacgtaccggcgataaagatctggtgacacacgaagtaacctggcctgattggtctactgtcgctgggcagcagacctccgtggtgacgtcaccggcgctcaaaggctacacggccgacaccaacgaaatccccgcaattacctatcatgcgggagatagcgacgttacgtacgtcgtcaaatataactaatag

SEQ ID NO: 8 - protein, 201 aa, Artificial Sequence
  fragment of mucin-binding protein from Lactobacillus reuteri
MHHHHHHGGSGMQEAQAIFYDETTGKEISGTREIATGKTDETISFTKDPNEVVKELEKQGYVFDKDNAKNNVFVAGTAYDKNSEVHQYFKYYLKHGHATVTPDQDPQKGQKTVTQTIKYEYADGTATGLADNVQTLTFKRTGDKDLVTHEVTWPDWSTVAGQQTSVVTSPALKGYTADTNEIPAITYHAGDSDVTYVVKYN

SEQ ID NO: 9 - DNA, 336 bp, Artificial Sequence
  fragment of mucin-binding protein from Lactobacillus acidophilus
atgcatcaccatcaccatcacggtggcagcggtatgaaagtggtgaatcaggtcattcattatatctacgaagatggtagtaccgcgcgtccggaccacgttagcgtaaagctggtgtttacacaaacgggcgtcaagaacttagccaccaacacgacgaattggaatggagagtggaccaagacccagaccttcgttgttgtaaaatcgccagaaattgaagggtatactgcagatcgcgaggaagtcggtccgtacgacatcacggtgaccaacgagaactataacgataaactggacaaagaagatactgtgatttaccgcgctaactaatag

SEQ ID NO: 10 - protein, 110 aa, Artificial Sequence
  fragment of mucin-binding protein from Lactobacillus acidophilus
MHHHHHHGGSGMKVVNQVIHYIYEDGSTARPDHVSVKLVFTQTGVKNLATNTTNWNGEWTKTQTFVVVKSPEIEGYTADREEVGPYDITVTNENYNDKLDKEDTVIYRAN

SEQ ID NO: 11 - DNA, 1377 bp, Artificial Sequence
  fragment of mucin-binding protein from Lactobacillus reuteri fused to Enhanced Green Fluorescent Protein
atgcaccatcatcatcatcatggcggtagcggcatggtaagcaaaggcgaagaattgtttacgggtgttgttccaatcctggttgaactggatggtgatgttaacggtcacaaatttagcgtgagcggcgaaggtgaaggcgatgcaacttatggcaaactgaccctgaaatttatttgcactacagggaaactgccggtgccgtggccaaccctggttaccaccctgacctatggcgttcagtgttttagccgctacccggaccacatgaagcagcacgattttttcaaaagcgcaatgccagaaggctatgtacaggaacgcaccatcttctttaaagatgatggcaactataaaacccgtgcggaagtcaaatttgaaggtgacaccctggttaatcgtattgaattgaaaggtattgattttaaagaagacggtaatatcctgggacataaactggagtataactataactcccacaatgtgtatatcatggcagataagcagaaaaacggcattaaggtgaatttcaaaattcgtcataatatcgaagatggtagtgtacagctggccgatcattaccagcagaataccccgatcggcgatggaccagtactgctgccggataatcattatctgagcacccagtcggctctgagcaaagatccgaacgaaaaacgcgaccacatggtactgctggagtttgttaccgccgctggtatcacactgggcatggatgagctgtataaatatggagggggcggctctggcggaggtggttccgggggtggcggatcgatgggtggcagcggtatggatgttcagcatgcggtgattaactatatcgacggcgaaagcgatgagattctgcacaccgacaaagtcaatggtcattccgatgaaaagatcaactactctacggccgatatgatcaaacaactcgaagcaaaagggtatgagctgtttaaagacaatttcccggctggcgaaaagtttgataacgacgatactaatgatcagttctacaccgtgatttttaaacaccaccgcgaaaacgtagacccaaatcatagttcggcggatggtacgaaaggcaccaagaccttgacagaaacggtccactataaatacgccaacggaactaaagcagcagaggatcagacagcccaagttaccttcacccgtaacggtgtgctggacgacgttacgggcattgtagcttgggggaaatggaatgaagcctcacagagctataaagctttaacgagtccgaccatcgcaggttatgcccctagcgaagctgtggtgaaacgctcgtccaactctgatgcagaacagggtccgaccctgaccgtcatttacactgccgactaatag

SEQ ID NO: 12 - protein, 457 aa, Artificial Sequence
  fragment of mucin-binding protein from Lactobacillus reuteri fused to Enhanced Green Fluorescent Protein
MHHHHHHGGSGMVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYKYGGGGSGGGGSGGGGSMGGSGMDVQHAVINYIDGESDEILHTDKVNGHSDEKINYSTADMIKQLEAKGYELFKDNFPAGEKFDNDDTNDQFYTVIFKHHRENVDPNHSSADGTKGTKTLTETVHYKYANGTKAAEDQTAQVTFTRNGVLDDVTGIVAWGKWNEASQSYKALTSPTIAGYAPSEAVVKRSSNSDAEQGPTLTVIYTAD

SEQ ID NO: 13 - DNA, 1371 bp, Artificial Sequence
  fragment of mucin-binding protein from Lactobacillus reuteri fused to Enhanced Green Fluorescent Protein
atgcaccatcatcatcatcatggcggtagcggcatggtaagcaaaggcgaagaattgtttacgggtgttgttccaatcctggttgaactggatggtgatgttaacggtcacaaatttagcgtgagcggcgaaggtgaaggcgatgcaacttatggcaaactgaccctgaaatttatttgcactacagggaaactgccggtgccgtggccaaccctggttaccaccctgacctatggcgttcagtgttttagccgctacccggaccacatgaagcagcacgattttttcaaaagcgcaatgccagaaggctatgtacaggaacgcaccatcttctttaaagatgatggcaactataaaacccgtgcggaagtcaaatttgaaggtgacaccctggttaatcgtattgaattgaaaggtattgattttaaagaagacggtaatatcctgggacataaactggagtataactataactcccacaatgtgtatatcatggcagataagcagaaaaacggcattaaggtgaatttcaaaattcgtcataatatcgaagatggtagtgtacagctggccgatcattaccagcagaataccccgatcggcgatggaccagtactgctgccggataatcattatctgagcacccagtcggctctgagcaaagatccgaacgaaaaacgcgaccacatggtactgctggagtttgttaccgccgctggtatcacactgggcatggatgagctgtataaatatggagggggcggctctggcggaggtggttccgggggtggcggatcgatgggtggcagcggtatgcagaaagttcatgtgcaatatattgatggcgaaaccgaccagatgctgcgccaggatgatcttgatggttacacggacgagacaatcccgtatagcactgcggaagggattaagaagtttgaaggcgatggttacgagctgttcaaagacaactttcctgccggcgagaaattcgacaatgatgataccaacgaccagttctacaccgtcatctttaaacaccaccgtgaaaatgtagatccgaaccatagtagtgcagacggtacgaaaggaaccaagacgttgactgaaaccgtgcactataaatatgctaatggcacaaaagcggcggaagatcaaaccgcccaggttacgtttacacgcaacggcgtgctggatgatgtcaccggtattgtagcatgggggaaatggaatgaagcctcccagtcgtacaaagcgttaacttcaccaaccatcgctggttatgcaccgtctgaagcggtggtgaaacgtagcagcaactctgacgccgaacaaggcccaacgctgaccgtcatttatacggctgactaatag

SEQ ID NO: 14 - protein, 455 aa, Artificial Sequence
  fragment of mucin-binding protein from Lactobacillus reuteri fused to Enhanced Green Fluorescent Protein
MHHHHHHGGSGMVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYKYGGGGSGGGGSGGGGSMGGSGMQKVHVQYIDGETDQMLRQDDLDGYTDETIPYSTAEGIKKFEGDGYELFKDNFPAGEKFDNDDTNDQFYTVIFKHHRENVDPNHSSADGTKGTKTLTETVHYKYANGTKAAEDQTAQVTFTRNGVLDDVTGIVAWGKWNEASQSYKALTSPTIAGYAPSEAVVKRSSNSDAEQGPTLTVIYTAD

SEQ ID NO: 15 - DNA, 1410 bp, Artificial Sequence
  fragment of mucin-binding protein from Lactobacillus reuteri fused to Enhanced Green Fluorescent Protein
atgcaccatcatcatcatcatggcggtagcggcatggtaagcaaaggcgaagaattgtttacgggtgttgttccaatcctggttgaactggatggtgatgttaacggtcacaaatttagcgtgagcggcgaaggtgaaggcgatgcaacttatggcaaactgaccctgaaatttatttgcactacagggaaactgccggtgccgtggccaaccctggttaccaccctgacctatggcgttcagtgttttagccgctacccggaccacatgaagcagcacgattttttcaaaagcgcaatgccagaaggctatgtacaggaacgcaccatcttctttaaagatgatggcaactataaaacccgtgcggaagtcaaatttgaaggtgacaccctggttaatcgtattgaattgaaaggtattgattttaaagaagacggtaatatcctgggacataaactggagtataactataactcccacaatgtgtatatcatggcagataagcagaaaaacggcattaaggtgaatttcaaaattcgtcataatatcgaagatggtagtgtacagctggccgatcattaccagcagaataccccgatcggcgatggaccagtactgctgccggataatcattatctgagcacccagtcggctctgagcaaagatccgaacgaaaaacgcgaccacatggtactgctggagtttgttaccgccgctggtatcacactgggcatggatgagctgtataaatatggagggggcggctctggcggaggtggttccgggggtggcggatcgatgggtggcagcggtatgcaagaagcggcgattaacttctatgatgaaaccggccataaactgctggacaatcagacgatccacttgaccggtaagactggcgagaaagtggatcgtacacaggcagatcagacgcttgccgacctggttaaacaaggttacgtcctcgacaaagaaaacaccgctaaagcatttccggccgatgcggtgtatgacaataatgatcagactccacaggagtttaccatttacctgaagcatgggacgacgcacacagatgctaccagcagcaaagccgatcagaagacggtatcggaaaccatccattacgtctataaagacggagttaacgcgaacaaaccggtggcagatgatgcgaataccaccgtgactttcaaacgcggctatacaaccgacaaagtaaccggcaagattgtctcctacgatccttggactgtggacggtaaacaagcggacagtaagacctttgatgccgtgaaatctccggttatcgctgggtatacggcagatcaagcggaagttgcagcccagacggtaactccggattcacagaacattaacaagaccgtctattatacagccgactaatag

SEQ ID NO: 16 - protein, 468 aa, Artificial Sequence
  fragment of mucin-binding protein from Lactobacillus reuteri fused to Enhanced Green Fluorescent Protein
MHHHHHHGGSGMVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYKYGGGGSGGGGSGGGGSMGGSGMQEAAINFYDETGHKLLDNQTIHLTGKTGEKVDRTQADQTLADLVKQGYVLDKENTAKAFPADAVYDNNDQTPQEFTIYLKHGTTHTDATSSKADQKTVSETIHYVYKDGVNANKPVADDANTTVTFKRGYTTDKVTGKIVSYDPWTVDGKQADSKTFDAVKSPVIAGYTADQAEVAAQTVTPDSQNINKTVYYTAD

SEQ ID NO: 17 - DNA, 1389 bp, Artificial Sequence
  fragment of mucin-binding protein from Lactobacillus reuteri fused to Enhanced Green Fluorescent Protein
atgcaccatcatcatcatcatggcggtagcggcatggtaagcaaaggcgaagaattgtttacgggtgttgttccaatcctggttgaactggatggtgatgttaacggtcacaaatttagcgtgagcggcgaaggtgaaggcgatgcaacttatggcaaactgaccctgaaatttatttgcactacagggaaactgccggtgccgtggccaaccctggttaccaccctgacctatggcgttcagtgttttagccgctacccggaccacatgaagcagcacgattttttcaaaagcgcaatgccagaaggctatgtacaggaacgcaccatcttctttaaagatgatggcaactataaaacccgtgcggaagtcaaatttgaaggtgacaccctggttaatcgtattgaattgaaaggtattgattttaaagaagacggtaatatcctgggacataaactggagtataactataactcccacaatgtgtatatcatggcagataagcagaaaaacggcattaaggtgaatttcaaaattcgtcataatatcgaagatggtagtgtacagctggccgatcattaccagcagaataccccgatcggcgatggaccagtactgctgccggataatcattatctgagcacccagtcggctctgagcaaagatccgaacgaaaaacgcgaccacatggtactgctggagtttgttaccgccgctggtatcacactgggcatggatgagctgtataaatatggagggggcggctctggcggaggtggttccgggggtggcggatcgatgggtggcagcggtatgcaggaagcacaagcgatcttctatgatgaaaccaccggcaaagagattagcggtacgcgcgaaattgccacagggaagactgacgagaccatctcgtttacgaaagatccgaacgaagtggtgaaagaactggagaaacagggctacgtctttgataaagacaatgcgaagaacaacgtatttgttgctggtaccgcctatgataagaatagtgaagtccatcagtacttcaaatattatttgaaacacggtcatgcgactgttaccccagaccaagacccgcagaaaggccagaagacggtgacacaaaccattaaatacgaatatgcagatggaactgccaccggtctggcggataatgttcagacgcttacgttcaaacgtaccggcgataaagatctggtgacacacgaagtaacctggcctgattggtctactgtcgctgggcagcagacctccgtggtgacgtcaccggcgctcaaaggctacacggccgacaccaacgaaatccccgcaattacctatcatgcgggagatagcgacgttacgtacgtcgtcaaatataactaatag

SEQ ID NO: 18 - protein, 461 aa, Artificial Sequence
  fragment of mucin-binding protein from Lactobacillus reuteri fused to Enhanced Green Fluorescent Protein
MHHHHHHGGSGMVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYKYGGGGSGGGGSGGGGSMGGSGMQEAQAIFYDETTGKEISGTREIATGKTDETISFTKDPNEVVKELEKQGYVFDKDNAKNNVFVAGTAYDKNSEVHQYFKYYLKHGHATVTPDQDPQKGQKTVTQTIKYEYADGTATGLADNVQTLTFKRTGDKDLVTHEVTWPDWSTVAGQQTSVVTSPALKGYTADTNEIPAITYHAGDSDVTYVVKYN

SEQ ID NO: 19 - DNA, 1116 bp, Artificial Sequence
  fragment of mucin-binding protein from Lactobacillus acidophilus fused to Enhanced Green Fluorescent Protein
atgcaccatcatcatcatcatggcggtagcggcatggtaagcaaaggcgaagaattgtttacgggtgttgttccaatcctggttgaactggatggtgatgttaacggtcacaaatttagcgtgagcggcgaaggtgaaggcgatgcaacttatggcaaactgaccctgaaatttatttgcactacagggaaactgccggtgccgtggccaaccctggttaccaccctgacctatggcgttcagtgttttagccgctacccggaccacatgaagcagcacgattttttcaaaagcgcaatgccagaaggctatgtacaggaacgcaccatcttctttaaagatgatggcaactataaaacccgtgcggaagtcaaatttgaaggtgacaccctggttaatcgtattgaattgaaaggtattgattttaaagaagacggtaatatcctgggacataaactggagtataactataactcccacaatgtgtatatcatggcagataagcagaaaaacggcattaaggtgaatttcaaaattcgtcataatatcgaagatggtagtgtacagctggccgatcattaccagcagaataccccgatcggcgatggaccagtactgctgccggataatcattatctgagcacccagtcggctctgagcaaagatccgaacgaaaaacgcgaccacatggtactgctggagtttgttaccgccgctggtatcacactgggcatggatgagctgtataaatatggagggggcggctctggcggaggtggttccgggggtggcggatcgatgggtggcagcggtatgaaagtggtgaatcaggtcattcattatatctacgaagatggtagtaccgcgcgtccggaccacgttagcgtaaagctggtgtttacacaaacgggcgtcaagaacttagccaccaacacgacgaattggaatggagagtggaccaagacccagaccttcgttgttgtaaaatcgccagaaattgaagggtatactgcagatcgcgaggaagtcggtccgtacgacatcacggtgaccaacgagaactataacgataaactggacaaagaagatactgtgatttaccgcgctaactaatag

SEQ ID NO: 20 - protein, 370 aa, Artificial Sequence
  fragment of mucin-binding protein from Lactobacillus acidophilus fused to Enhanced Green Fluorescent Protein
MHHHHHHGGSGMVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYKYGGGGSGGGGSGGGGSMGGSGMKVVNQVIHYIYEDGSTARPDHVSVKLVFTQTGVKNLATNTTNWNGEWTKTQTFVVVKSPEIEGYTADREEVGPYDITVTNENYNDKLDKEDTVIYRAN

SEQ ID NO: 21 - DNA, 2376 bp, Artificial Sequence
  fragment of mucin-binding protein from Lactobacillus reuteri fused to a variant of Tyrosine Ammonia Lyase from Stanieria cyanosphaera
atgcaccatcatcatcatcatggcggtagcggcatgggtggcagcggtatgcaagaagcggcgattaacttctatgatgaaaccggccataaactgctggacaatcagacgatccacttgaccggtaagactggcgagaaagtggatcgtacacaggcagatcagacgcttgccgacctggttaaacaaggttacgtcctcgacaaagaaaacaccgctaaagcatttccggccgatgcggtgtatgacaataatgatcagactccacaggagtttaccatttacctgaagcatgggacgacgcacacagatgctaccagcagcaaagccgatcagaagacggtatcggaaaccatccattacgtctataaagacggagttaacgcgaacaaaccggtggcagatgatgcgaataccaccgtgactttcaaacgcggctatacaaccgacaaagtaaccggcaagattgtctcctacgatccttggactgtggacggtaaacaagcggacagtaagacctttgatgccgtgaaatctccggttatcgctgggtatacggcagatcaagcggaagttgcagcccagacggtaactccggattcacagaacattaacaagaccgtctattatacagccgacggagggggcggctctggcggaggtggttccgggggtggcggatcgatgatcattgctagccagaccgataccttcgacagcaaaagtccgcaccaggccagcgacagcaccgttacggtgggtgataagccgctgacgatcaatgaggtgtcggctgttgcgcgttatggtgcgaaggttcgtctgaccgacaaagaggaagtgctgcagcgtgttaacgcgagctgcgagtgggtggcgcaggcggttgcgaagggtgaaccgatttacggcgttaccactggtttcggtggcatggcgaacgtggttatcaccccggaccaggcgaaggagctgcagaataacctgccacgctatcacaaagcgggtgcgggccaaaagctgccgctggcggatgttcgtgcgggtatgctgctgcgtgcgaacagccacctgcacggtgcgagcggtatccgtctggagctgattcagcgtattgaaaccttcctgaacgcaggcgtgaccccgcacgtttgcgaatttggtagcatcggtgcgagcggtgacctgaccccgctggcgtacatcaccggtgcgctggttggtctgaacgacagttataccgttgattttaacggcgaggaaatgaccgcgccggaggcgctgaaacgcctgggtctggagccgctggaactgctgccgaaggaaggcctggcgatgatgaacggtaccgcggtgatgaccggtattgcggcgaactgcgtttatgatgcgcagaacctgctggcgctgagcctgggtgcgcatgcgctggcgctgcagggcctgaacggtaccaaccagagcttccacccgtttattcaccagtgcaaaccgcacccgggtcaaatttggagcgcgcagcaaatgctggagctgctgcagggcagcctgctgatccgtgacgagctggatggtagccacgactaccgcggcgaagatccgatccaagatcgttacagcatgcgttgcctgccgcagtataccggcccgattgtcgacggtattgcagaaatcgcgaatcaaatcgaggtcgaaatcaacagcgtgaccgacaacccgctgatcgatgttgcggggaacgcgagctaccacggtggcaacttcatgggccagtatattggtaccagcatggaccgtctgcgttactatctgggcctgctggcgaaacacctggatgtgcaaatcgcgctgctgaccatgccggagtttaacaacggtctgccggctagcctggtgggtaacaccgaccgcatggttaacatgggcctgaagggtctgcagattaccggtaacagcatcatgccgctgctgaccttcctgggcaacactctgaccgatcgtttcccgacccacgcggagcagttcaaccaaaacatcaacagccagggttttggcagcgcgaacctgacccgtcaaagcattgaaatcttccagcaatatattgcgatcgcgctgatgtttggtgtgcaaagcgttgacctgcgtaccaagaaaattgcgaaccactacgatgcgagccaatgcctgagcccggccacccgccaactgtatctggcggtcaaggaagtggttggtaagactccgagcaaagatcgtccgtatatctggaacgacaacgagcaggcgctggatgaacacattgcgctgatcgcggaggatatcactaaagatggccgtattgttcaggcgatcaaccaagtggttagcagttaatag

SEQ ID NO: 22 - protein, 790 aa, Artificial Sequence
  fragment of mucin-binding protein from Lactobacillus reuteri fused to a variant of Tyrosine Ammonia Lyase from Stanieria cyanosphaera
MHHHHHHGGSGMGGSGMQEAAINFYDETGHKLLDNQTIHLTGKTGEKVDRTQADQTLADLVKQGYVLDKENTAKAFPADAVYDNNDQTPQEFTIYLKHGTTHTDATSSKADQKTVSETIHYVYKDGVNANKPVADDANTTVTFKRGYTTDKVTGKIVSYDPWTVDGKQADSKTFDAVKSPVIAGYTADQAEVAAQTVTPDSQNINKTVYYTADGGGGSGGGGSGGGGSMIIASQTDTFDSKSPHQASDSTVTVGDKPLTINEVSAVARYGAKVRLTDKEEVLQRVNASCEWVAQAVAKGEPIYGVTTGFGGMANVVITPDQAKELQNNLPRYHKAGAGQKLPLADVRAGMLLRANSHLHGASGIRLELIQRIETFLNAGVTPHVCEFGSIGASGDLTPLAYITGALVGLNDSYTVDFNGEEMTAPEALKRLGLEPLELLPKEGLAMMNGTAVMTGIAANCVYDAQNLLALSLGAHALALQGLNGTNQSFHPFIHQCKPHPGQIWSAQQMLELLQGSLLIRDELDGSHDYRGEDPIQDRYSMRCLPQYTGPIVDGIAEIANQIEVEINSVTDNPLIDVAGNASYHGGNFMGQYIGTSMDRLRYYLGLLAKHLDVQIALLTMPEFNNGLPASLVGNTDRMVNMGLKGLQITGNSIMPLLTFLGNTLTDRFPTHAEQFNQNINSQGFGSANLTRQSIEIFQQYIAIALMFGVQSVDLRTKKIANHYDASQCLSPATRQLYLAVKEVVGKTPSKDRPYIWNDNEQALDEHIALIAEDITKDGRIVQAINQVVSS

SEQ ID NO: 23 - DNA, 2082 bp, Artificial Sequence
  fragment of mucin-binding protein from Lactobacillus acidophilus fused to a variant of Tyrosine Ammonia Lyase from Stanieria cyanosphaera
atgcaccatcatcatcatcatggcggtagcggcatgggtggcagcggtatgaaagtggtgaatcaggtcattcattatatctacgaagatggtagtaccgcgcgtccggaccacgttagcgtaaagctggtgtttacacaaacgggcgtcaagaacttagccaccaacacgacgaattggaatggagagtggaccaagacccagaccttcgttgttgtaaaatcgccagaaattgaagggtatactgcagatcgcgaggaagtcggtccgtacgacatcacggtgaccaacgagaactataacgataaactggacaaagaagatactgtgatttaccgcgctaacggagggggcggctctggcggaggtggttccgggggtggcggatcgatgatcattgctagccagaccgataccttcgacagcaaaagtccgcaccaggccagcgacagcaccgttacggtgggtgataagccgctgacgatcaatgaggtgtcggctgttgcgcgttatggtgcgaaggttcgtctgaccgacaaagaggaagtgctgcagcgtgttaacgcgagctgcgagtgggtggcgcaggcggttgcgaagggtgaaccgatttacggcgttaccactggtttcggtggcatggcgaacgtggttatcaccccggaccaggcgaaggagctgcagaataacctgccacgctatcacaaagcgggtgcgggccaaaagctgccgctggcggatgttcgtgcgggtatgctgctgcgtgcgaacagccacctgcacggtgcgagcggtatccgtctggagctgattcagcgtattgaaaccttcctgaacgcgggcgtgaccccgcacgtttgcgaatttggtagcatcggtgcgagcggtgacctgaccccgctggcgtacatcaccggtgcgctggttggtctgaacgacagttataccgttgattttaacggcgaggaaatgaccgcgccggaggcgctgaaacgcctgggtctggagccgctggaactgctgccgaaggaaggcctggcgatgatgaacggtaccgcggtgatgaccggtattgcggcgaactgcgtttatgatgcgcagaacctgctggcgctgagcctgggtgcgcatgcgctggcgctgcagggcctgaacggtaccaaccagagcttccacccgtttattcaccagtgcaaaccgcacccgggtcaaatttggagcgcgcagcaaatgctggagctgctgcagggcagcctgctgatccgtgacgagctggatggtagccacgactaccgcggcgaagatccgatccaagatcgttacagcatgcgttgcctgccgcagtataccggcccgattgtcgacggtattgcagaaatcgcgaatcaaatcgaggtcgaaatcaacagcgtgaccgacaacccgctgatcgatgttgcggggaacgcgagctaccacggtggcaacttcatgggccagtatattggtaccagcatggaccgtctgcgttactatctgggcctgctggcgaaacacctggatgtgcaaatcgcgctgctgaccatgccggagtttaacaacggtctgccggctagcctggtgggtaacaccgaccgcatggttaacatgggcctgaagggtctgcagattaccggtaacagcatcatgccgctgctgaccttcctgggcaacactctgaccgatcgtttcccgacccacgcggagcagttcaaccaaaacatcaacagccagggttttggcagcgcgaacctgacccgtcaaagcattgaaatcttccagcaatatattgcgatcgcgctgatgtttggtgtgcaaagcgttgacctgcgtaccaagaaaattgcgaaccactacgatgcgagccaatgcctgagcccggccacccgccaactgtatctggcggtcaaggaagtggttggtaagactccgagcaaagatcgtccgtatatctggaacgacaacgagcaggcgctggatgaacacattgcgctgatcgcggaggatatcactaaagatggccgtattgttcaggcgatcaaccaagtggttagcagttaatag

SEQ ID NO: 24 - protein, 692 aa, Artificial Sequence
  fragment of mucin-binding protein from Lactobacillus acidophilus fused to a variant of Tyrosine Ammonia Lyase from Stanieria cyanosphaera
MHHHHHHGGSGMGGSGMKVVNQVIHYIYEDGSTARPDHVSVKLVFTQTGVKNLATNTTNWNGEWTKTQTFVVVKSPEIEGYTADREEVGPYDITVTNENYNDKLDKEDTVIYRANGGGGSGGGGSGGGGSMIIASQTDTFDSKSPHQASDSTVTVGDKPLTINEVSAVARYGAKVRLTDKEEVLQRVNASCEWVAQAVAKGEPIYGVTTGFGGMANVVITPDQAKELQNNLPRYHKAGAGQKLPLADVRAGMLLRANSHLHGASGIRLELIQRIETFLNAGVTPHVCEFGSIGASGDLTPLAYITGALVGLNDSYTVDFNGEEMTAPEALKRLGLEPLELLPKEGLAMMNGTAVMTGIAANCVYDAQNLLALSLGAHALALQGLNGTNQSFHPFIHQCKPHPGQIWSAQQMLELLQGSLLIRDELDGSHDYRGEDPIQDRYSMRCLPQYTGPIVDGIAEIANQIEVEINSVTDNPLIDVAGNASYHGGNFMGQYIGTSMDRLRYYLGLLAKHLDVQIALLTMPEFNNGLPASLVGNTDRMVNMGLKGLQITGNSIMPLLTFLGNTLTDRFPTHAEQFNQNINSQGFGSANLTRQSIEIFQQYIAIALMFGVQSVDLRTKKIANHYDASQCLSPATRQLYLAVKEVVGKTPSKDRPYIWNDNEQALDEHIALIAEDITKDGRIVQAINQVVSS

SEQ ID NO: 25 - DNA, 1686 bp, Artificial Sequence
  variant of Tyrosine Ammonia Lyase from Stanieria cyanosphaera
atgatcattgctagccagaccgataccttcgacagcaaaagtccgcaccaggccagcgacagcaccgttacggtgggtgataagccgctgacgatcaatgaggtgtcggctgttgcgcgttatggtgcgaaggttcgtctgaccgacaaagaggaagtgctgcagcgtgttaacgcgagctgcgagtgggtggcgcaggcggttgcgaagggtgaaccgatttacggcgttaccactggtttcggtggcatggcgaacgtggttatcaccccggaccaggcgaaggagctgcagaataacctgccacgctatcacaaagcgggtgcgggccaaaagctgccgctggcggatgttcgtgcgggtatgctgctgcgtgcgaacagccacctgcacggtgcgagcggtatccgtctggagctgattcagcgtattgaaaccttcctgaacgcgggcgtgaccccgcacgtttgcgaatttggtagcatcggtgcgagcggtgacctgaccccgctggcgtacatcaccggtgcgctggttggtctgaacgacagttataccgttgattttaacggcgaggaaatgaccgcgccggaggcgctgaaacgcctgggtctggagccgctggaactgctgccgaaggaaggcctggcgatgatgaacggtaccgcggtgatgaccggtattgcggcgaactgcgtttatgatgcgcagaacctgctggcgctgagcctgggtgcgcatgcgctggcgctgcagggcctgaacggtaccaaccagagcttccacccgtttattcaccagtgcaaaccgcacccgggtcaaatttggagcgcgcagcaaatgctggagctgctgcagggcagcctgctgatccgtgacgagctggatggtagccacgactaccgcggcgaagatccgatccaagatcgttacagcatgcgttgcctgccgcagtataccggcccgattgtcgacggtattgcagaaatcgcgaatcaaatcgaggtcgaaatcaacagcgtgaccgacaacccgctgatcgatgttgcggggaacgcgagctaccacggtggcaacttcatgggccagtatattggtaccagcatggaccgtctgcgttactatctgggcctgctggcgaaacacctggatgtgcaaatcgcgctgctgaccatgccggagtttaacaacggtctgccggctagcctggtgggtaacaccgaccgcatggttaacatgggcctgaagggtctgcagattaccggtaacagcatcatgccgctgctgaccttcctgggcaacactctgaccgatcgtttcccgacccacgcggagcagttcaaccaaaacatcaacagccagggttttggcagcgcgaacctgacccgtcaaagcattgaaatcttccagcaatatattgcgatcgcgctgatgtttggtgtgcaaagcgttgacctgcgtaccaagaaaattgcgaaccactacgatgcgagccaatgcctgagcccggccacccgccaactgtatctggcggtcaaggaagtggttggtaagactccgagcaaagatcgtccgtatatctggaacgacaacgagcaggcgctggatgaacacattgcgctgatcgcggaggatatcactaaagatggccgtattgttcaggcgatcaaccaagtggttagcagt

SEQ ID NO: 26 - protein, 562 aa, Artificial Sequence
  variant of Tyrosine Ammonia Lyase from Stanieria cyanosphaera
MIIASQTDTFDSKSPHQASDSTVTVGDKPLTINEVSAVARYGAKVRLTDKEEVLQRVNASCEWVAQAVAKGEPIYGVTTGFGGMANVVITPDQAKELQNNLPRYHKAGAGQKLPLADVRAGMLLRANSHLHGASGIRLELIQRIETFLNAGVTPHVCEFGSIGASGDLTPLAYITGALVGLNDSYTVDFNGEEMTAPEALKRLGLEPLELLPKEGLAMMNGTAVMTGIAANCVYDAQNLLALSLGAHALALQGLNGTNQSFHPFIHQCKPHPGQIWSAQQMLELLQGSLLIRDELDGSHDYRGEDPIQDRYSMRCLPQYTGPIVDGIAEIANQIEVEINSVTDNPLIDVAGNASYHGGNFMGQYIGTSMDRLRYYLGLLAKHLDVQIALLTMPEFNNGLPASLVGNTDRMVNMGLKGLQITGNSIMPLLTFLGNTLTDRFPTHAEQFNQNINSQGFGSANLTRQSIEIFQQYIAIALMFGVQSVDLRTKKIANHYDASQCLSPATRQLYLAVKEVVGKTPSKDRPYIWNDNEQALDEHIALIAEDITKDGRIVQAINQVVSS